(19) United States
(12) Patent Application Publication
(10) Pub. No.: US 2026/0369425 A1
(43) Pub. Date: Nov. 19, 2026

(54) Disease Prediction from Large-Scale Genotype Databases

(71) Applicant: 23andMe Genomics LLC, Los Altos, CA (US)

(72) Inventors: Aly Azeem Khan, Chicago, IL (US); Payam Dibaeinia, Chicago, IL (US)

(21) Appl. No.: 19/648,205

(22) Filed: Apr. 15, 2026

Related U.S. Application Data
(60) Provisional application No. 63/806,145, filed on May 15, 2025.

(51) Int. Cl. G16H 50/70; G06N 3/045; G16H 50/20

(52) U.S. Cl. CPC G16H 50/70; G06N 3/045; G16H 50/20

(57) ABSTRACT

An embodiment may involve: obtaining a genetic dataset for a plurality of individuals, wherein the genetic dataset includes: representations of presence or absence of a set of genetic variants at specific loci for each of the individuals, and further representations of presence or absence of a plurality of health conditions exhibited by each of the individuals; and training a machine learning model with the genetic dataset, wherein the machine learning model includes: a variant embedding layer that projects each of the representations of presence or absence of the set of genetic variants into respective multidimensional vectors based on variant-specific embedding functions, one or more layers of transformers with neighborhood attention that process the respective multidimensional vectors, and a further layer that maps output of the one or more layers of transformers into predictions of presence or absence of the plurality of health conditions.

### CROSS-REFERENCE TO RELATED APPLICATION

This application claims priority to U.S. provisional patent application No. 63/806,145, filed May 15, 2025, which is hereby incorporated by reference in its entirety.

### BACKGROUND

Developing predictive models from high-dimensional, complex structured data is a fundamental machine learning challenge. This challenge is acutely relevant in modern genomics, where biobanks are rapidly scaling toward million-sample sizes (N>1,000,000) and individual genomes are characterized by hundreds of thousands to millions of genetic variants (e.g., single nucleotide polymorphisms, SNPs), which is a regime of ultra-high dimensionality (L>100,000). Effectively leveraging individual-level genomic data at this unprecedented N×L scale can unlock deeper insights into complex trait genetics, such as predicting disease susceptibility.

Current state-of-the-art methods for disease risk prediction primarily rely on polygenic risk scores (PRS) derived from genome-wide association study (GWAS) summary statistics. While effective to a degree, these methods are predominantly linear, capturing additive genetic effects. Furthermore, by operating on summary statistics, they discard potentially valuable individual-level haplotype information and struggle to model non-additive genetic interactions (epistasis). These limitations may cap predictive performance, especially as dataset sizes scale to where subtle interaction effects might become detectable.

### SUMMARY

Transformer architectures have revolutionized sequence modeling in other domains by capturing complex, long-range dependencies via self-attention. The attention mechanism provides a powerful inductive tool for genomics, enabling more effective modeling of pairwise linear and non-linear interactions between genetic loci compared to traditional architectures or inherently linear models. However, a barrier persists: the prohibitive O(L²) computational complexity of standard self-attention renders its direct application to genome-scale sequences (L>100,000) computationally infeasible. Addressing this scalability bottleneck is a pathway to harnessing the power of transformers for large-scale genomics.

The embodiments herein employ a transformer-based architecture specifically engineered for scalable, end-to-end, multitask disease risk prediction directly from ultra-high-dimensional (L>100,000) individual-level genotypes (N>1,000,000). A significant technical improvement lies in its scalability, achieved by incorporating neighborhood attention (NA), an efficient attention mechanism restricting computations to local genomic windows, resulting in O(L) complexity per layer. This design aligns with the biological structure of the genome, which is treated as a series of linked regions, called linkage disequilibrium (LD) blocks, where genetic variants are often inherited together. These embodiments stack NA layers, which first model interactions within LD blocks, then progressively integrate information between neighboring LD blocks in deeper layers, capturing larger-scale genetic patterns influencing disease.

An example embodiment involves: obtaining a genetic dataset for a plurality of individuals, wherein the genetic dataset includes: representations of presence or absence of a set of genetic variants at specific loci for each of the individuals, and further representations of presence or absence of a plurality of health conditions exhibited by each of the individuals; and training a machine learning model with the genetic dataset, wherein the machine learning model includes: a variant embedding layer that projects each of the representations of presence or absence of the set of genetic variants into respective multidimensional vectors based on variant-specific embedding functions, one or more layers of transformers with neighborhood attention that process the respective multidimensional vectors, and a further layer that maps output of the one or more layers of transformers into predictions of presence or absence of the plurality of health conditions.

A further related example embodiment involves: obtaining a particular genetic dataset for an individual, wherein the particular genetic dataset includes representations of presence or absence of a set of genetic variants at specific loci for the individual; providing the particular genetic dataset to a trained machine learning model, wherein the trained machine learning model includes: a variant embedding layer that projects each of the representations of presence or absence of the set of genetic variants into respective multidimensional vectors based on variant-specific embedding functions, one or more layers of transformers with neighborhood attention that process the respective multidimensional vectors, and a further layer that maps output of the one or more layers of transformers into predictions of presence or absence of a plurality of health conditions; and obtaining from the trained machine learning model, the predictions of presence or absence of the plurality of health conditions for the individual.

These, as well as other embodiments, aspects, advantages, and alternatives, will become apparent to those of ordinary skill in the art by reading the following detailed description, with reference where appropriate to the accompanying drawings. Further, this summary and other descriptions and figures provided herein are intended to illustrate embodiments by way of example only and, as such, that numerous variations are possible. For instance, structural elements and process steps can be rearranged, combined, distributed, eliminated, or otherwise changed, while remaining within the scope of the embodiments as claimed.

### BRIEF DESCRIPTION OF THE DRAWINGS

FIG. 1 illustrates a schematic drawing of a computing device, in accordance with example embodiments.

FIG. 2 illustrates a schematic drawing of a server device cluster, in accordance with example embodiments.

FIG. 3 illustrates a schematic drawing of PRSformer and its possible components, in accordance with example embodiments.

FIGS. 4A and 4B illustrate benchmarking PRSformer against baseline methods using area under the receiver operating characteristic (AUROC), in accordance with example embodiments.

FIG. 5 illustrates the impact of training scale on non-linear model advantage, in accordance with example embodiments.

FIG. 6 illustrates multitask versus single-task training for Celiac disease, in accordance with example embodiments.

FIG. 7 illustrates AUROC of European-only PRSformer versus multi-ancestry PRSformer-ME on a multi-ancestry test set, in accordance with example embodiments.

FIGS. 8A-1, 8A-2, and 8A-3 illustrate benchmarking PRSformer against baseline methods using area under the precision-recall curve (AUPRC), in accordance with example embodiments.

FIGS. 8B-1 and 8B-2 illustrate benchmarking of models trained on the combined training and validation datasets, in accordance with example embodiments.

FIGS. 8C-1 and 8C-2 illustrate prediction performance across different training scales for the non-linear model and linear baseline, both trained in a multitask setting using the same input variant set as PRSformer, in accordance with example embodiments.

FIGS. 8D-1 and 8D-2 illustrate prediction performance across different training scales for the non-linear model and linear baseline, both trained in a multitask setting using subsetted variant sets at each downsampled scale, in accordance with example embodiments.

FIG. 8E illustrates comparison of multitask versus single-task training for three additional diseases across different training scales, all using the same input variant set as PRSformer, in accordance with example embodiments.

FIGS. 8F-1 and 8F-2 illustrate comparison of multitask versus single-task training for four tested diseases across different training scales, in accordance with example embodiments.

FIG. 9A illustrates the number of selected variants per disease across European datasets, in accordance with example embodiments.

FIG. 9B illustrates training sample sizes (case/control) per disease across European and multi-ancestry datasets, in accordance with example embodiments.

FIG. 9C illustrates test data sample sizes (case/control) per disease across ancestry groups, in accordance with example embodiments.

FIG. 9D illustrates validation dataset sample sizes (case/control) per disease (only European individuals), in accordance with example embodiments.

FIG. 9E illustrates tuning of the training steps based on validation AUROC, in accordance with example embodiments.

FIG. 9F illustrates tuning of attention heads and model dimension based on validation AUROC, in accordance with example embodiments.

FIG. 9G illustrates tuning of attention dilation and number of transformer blocks based on validation AUROC, in accordance with example embodiments.

FIG. 9H illustrates tuning of the window size of neighborhood attention based on validation AUROC, in accordance with example embodiments.

FIG. 9I illustrates the number of selected variants per disease across non-European ancestries, in accordance with example embodiments.

FIG. 9J illustrates characteristics of multitask models trained on downsampled datasets with subsetted input variants, in accordance with example embodiments.

FIG. 10 is a flow chart, in accordance with example embodiments.

FIG. 11 is a flow chart, in accordance with example embodiments.

### DETAILED DESCRIPTION

Example methods, devices, and systems are described herein. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features unless stated as such. Thus, other embodiments can be utilized and other changes can be made without departing from the scope of the subject matter presented herein.

Accordingly, the example embodiments described herein are not meant to be limiting. It will be readily understood that the aspects of the present disclosure, as generally described herein, and illustrated in the figures, can be arranged, substituted, combined, separated, and designed in a wide variety of different configurations. For example, the separation of software features into “client” and “server” components may occur in a number of ways.

Further, unless context suggests otherwise, the features illustrated in each of the figures may be used in combination with one another. Thus, the figures should be generally viewed as component aspects of one or more overall embodiments, with the understanding that not all illustrated features are necessary for each embodiment.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

Unless clearly indicated otherwise herein, the term “or” is to be interpreted as the inclusive disjunction. For example, the phrase “A, B, or C” is true if any one or more of the arguments A, B, C are true, and is only false if all of A, B, and C are false.

### I. Definitions

The following definitions are provided to clarify terms as they are used in this application. These definitions are intended to aid in understanding the invention but should not be considered limiting. Unless otherwise specified, terms should be interpreted in a manner consistent with their ordinary meaning in the relevant technical field. In cases where a term has multiple meanings, the interpretation most applicable to this disclosure should be used.

A genome is the complete set of genetic material present in an organism, including all of its deoxyribonucleic acid (DNA) and/or ribonucleic acid (RNA). It includes the full sequence of nucleotides that encode the instructions necessary for the growth, development, functioning, and reproduction of the organism. The genome includes both coding regions, which contain genes responsible for protein synthesis, and non-coding regions, which may regulate gene expression and other cellular functions.

Linkage disequilibrium (LD) describes a situation in which specific versions of genes at different positions on a chromosome tend to be inherited together more often than would be expected if their inheritances were driven solely by chance. In a freely mixing population with sufficient recombination, each version of a gene (“allele”) at one location would pair randomly with alleles at other locations. However, when certain combinations appear more (or less) frequently than random assortment predicts-due to factors like limited recombination between nearby sites, natural selection favoring particular allele pairs, or the mixing of distinct populations-those alleles are in linkage disequilibrium.

The transformer architecture is a type of neural network machine learning model that processes sequential data through a mechanism known as self-attention, which allows the model to weigh the relevance of each token in an input sequence relative to every other token, regardless of positional distance. Unlike recurrent architectures that process tokens sequentially, the transformer operates on the entire sequence in parallel, computing attention scores through learned query, key, and value projections. These projections are organized into multiple parallel attention heads, enabling the model to capture diverse relational patterns simultaneously. The outputs of the multi-head attention layers may be combined with residual connections and layer normalization, then passed through position-wise feed-forward networks to produce contextualized representations of the input.

In its canonical form, the transformer comprises an encoder-decoder structure, where the encoder generates a contextual representation of an input sequence and the decoder autoregressively produces an output sequence while attending to both its own prior outputs and the encoder's representations via cross-attention. Positional encodings are added to the input embeddings to supply the model with sequence-order information that the attention mechanism might not inherently capture. The architecture's reliance on matrix-multiplication-based attention computations makes it highly amenable to parallel processing on modern hardware accelerators.

Transformers with neighborhood attention use a sliding-window strategy in place of the standard global attention mechanism that compares every token to every other token (a process that scales quadratically). With neighborhood attention, each token only gathers context from a fixed number of its immediate neighbors, dramatically reducing the number of pairwise comparisons that the model performs. Despite this locality, stacking multiple layers of these transformers still allows information to flow across the entire sequence, since each layer's window shifts slightly, gradually extending the model's effective reach.

### II. Example Computing Devices and Cloud-Based Computing Environments

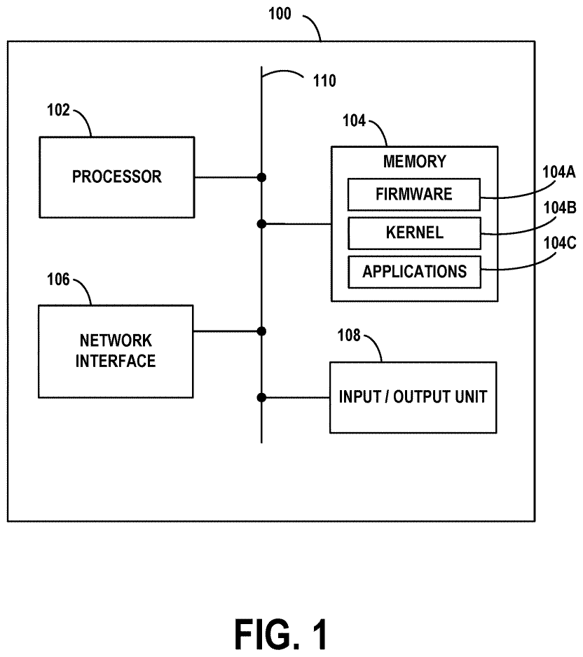

FIG. 1 is a simplified block diagram exemplifying a computing device 100, illustrating some of the components that could be included in a computing device arranged to operate in accordance with the embodiments herein. Computing device 100 could be a client device (e.g., a device actively operated by a user), a server device (e.g., a device that provides computational services to client devices), or some other type of computational platform. Some server devices may operate as client devices from time to time in order to perform particular operations, and some client devices may incorporate server features.

In this example, computing device 100 includes processor 102, memory 104, network interface 106, and input/output unit 108, all of which may be coupled by system bus 110 or a similar mechanism. In some embodiments, computing device 100 may include other components and/or peripheral devices (e.g., detachable storage, printers, and so on).

Processor 102 may be one or more of any type of computer processing element, such as a central processing unit (CPU), a graphical processing unit (GPU), a digital signal processor (DSP), a network processor, an encryption processor, and/or a form of integrated circuit or controller that performs processor operations. In some cases, processor 102 may be one or more single-core processors. In other cases, processor 102 may be one or more multi-core processors with multiple independent processing units. Processor 102 may also include register memory for temporarily storing instructions being executed and related data, as well as cache memory for temporarily storing recently used instructions and data.

GPUs, in particular, have grown in importance. They include specialized circuitry designed to perform rapid mathematical calculations for rendering graphics, processing large datasets, and supporting machine learning. A GPU typically consists of hundreds or thousands of small cores that operate simultaneously, facilitating the decomposition of tasks into smaller, more manageable pieces that are processed in parallel. This parallelism allows GPUs to be significantly faster than traditional CPUs for certain types of calculations.

Memory 104 may be any form of computer-usable memory, including but not limited to random access memory (RAM), read-only memory (ROM), and non-volatile memory (e.g., flash memory, hard disk drives, solid state drives, compact discs (CDs), digital video discs (DVDs), and/or tape storage). Thus, memory 104 represents both main memory units, as well as long-term storage. Herein, any non-volatile memory may be referred to as persistent storage.

Memory 104 may store program instructions and/or data on which program instructions may operate. By way of example, memory 104 may store these program instructions on a non-transitory, computer-readable medium, such that the instructions are executable by processor 102 to carry out any of the methods, processes, or operations disclosed in this specification or the accompanying drawings.

As shown in FIG. 1, memory 104 may include firmware 104A, kernel 104B, and/or applications 104C. Firmware 104A may be program code used to boot or otherwise initiate some or all of computing device 100. Kernel 104B may be an operating system, including modules for memory management, scheduling and management of processes, input/output, and communication. Kernel 104B may also include device drivers that allow the operating system to communicate with the hardware modules (e.g., memory units, networking interfaces, ports, and buses) of computing device 100. Applications 104C may be one or more user-space software programs, such as web browsers or email clients, as well as any software libraries used by these programs. Memory 104 may also store data used by these and other programs and applications.

Network interface 106 may take the form of one or more wireline interfaces, such as Ethernet (e.g., Fast Ethernet, Gigabit Ethernet, 10 Gigabit Ethernet, Ethernet over fiber, and so on). Network interface 106 may also support communication over one or more non-Ethernet media, such as coaxial cables or power lines, or over wide-area media, such as Synchronous Optical Networking (SONET), Synchronous Digital Hierarchy (SDH), Data Over Cable Service Interface Specification (DOCSIS), or other technologies. Network interface 106 may additionally take the form of one or more wireless interfaces, such as IEEE 802.11 (Wifi), BLUETOOTH®, global positioning system (GPS), or a wide-area wireless interface. However, other forms of physical layer interfaces and other types of standard or proprietary communication protocols may be used over network interface 106. Furthermore, network interface 106 may comprise multiple physical interfaces. For instance, some embodiments of computing device 100 may include Ethernet, BLUETOOTH®, and Wifi interfaces.

Input/output unit 108 may facilitate user and peripheral device interaction with computing device 100. Input/output unit 108 may include one or more types of input devices, such as a keyboard, a mouse, a touch screen, and so on. Similarly, input/output unit 108 may include one or more types of output devices, such as a screen, monitor, printer, and/or one or more light emitting diodes (LEDs). Additionally or alternatively, computing device 100 may communicate with other devices using a universal serial bus (USB) or high-definition multimedia interface (HDMI) port interface, for example.

In some embodiments, one or more computing devices like computing device 100 may be deployed. The exact physical location, connectivity, and configuration of these computing devices may be unknown and/or unimportant to client devices. Accordingly, the computing devices may be referred to as “cloud-based” devices that may be housed at various remote data center locations.

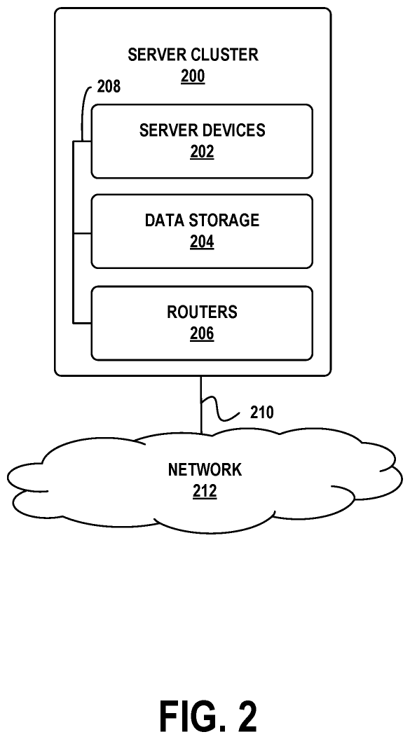

FIG. 2 depicts a cloud-based server cluster 200 in accordance with example embodiments. In FIG. 2, operations of a computing device (e.g., computing device 100) may be distributed between server devices 202, data storage 204, and routers 206, all of which may be connected by local cluster network 208. The number of server devices 202, data storages 204, and routers 206 in server cluster 200 may depend on the computing task(s) and/or applications assigned to server cluster 200.

For example, server devices 202 can be configured to perform various computing tasks of computing device 100. Thus, computing tasks can be distributed among one or more of server devices 202. To the extent that these computing tasks can be performed in parallel, such a distribution of tasks may reduce the total time to complete these tasks and return a result. For purposes of simplicity, both server cluster 200 and individual server devices 202 may be referred to as a “server device.” This nomenclature should be understood to imply that one or more distinct server devices, data storage devices, and cluster routers may be involved in server device operations.

Data storage 204 may be data storage arrays that include drive array controllers configured to manage read and write access to groups of hard disk drives and/or solid state drives. The drive array controllers, alone or in conjunction with server devices 202, may also be configured to manage backup or redundant copies of the data stored in data storage 204 to protect against drive failures or other types of failures that prevent one or more of server devices 202 from accessing units of data storage 204. Other types of memory aside from drives may be used.

Routers 206 may include networking equipment configured to provide internal and external communications for server cluster 200. For example, routers 206 may include one or more packet-switching and/or routing devices (including switches and/or gateways) configured to provide (i) network communications between server devices 202 and data storage 204 via local cluster network 208, and/or (ii) network communications between server cluster 200 and other devices via communication link 210 to network 212.

Additionally, the configuration of routers 206 can be based at least in part on the data communication requirements of server devices 202 and data storage 204, the latency and throughput of the local cluster network 208, the latency, throughput, and cost of communication link 210, and/or other factors that may contribute to the cost, speed, fault-tolerance, resiliency, efficiency, and/or other design goals of the system architecture.

As a possible example, data storage 204 may include any form of database, such as a structured query language (SQL) database or a No-SQL database (e.g., MongoDB). Various types of data structures may store the information in such a database, including but not limited to files, tables, arrays, lists, trees, and tuples. Furthermore, any databases in data storage 204 may be monolithic or distributed across multiple physical devices.

Server devices 202 may be configured to transmit data to and receive data from data storage 204. This transmission and retrieval may take the form of SQL queries or other types of database queries, and the output of such queries, respectively. Additional text, images, video, and/or audio may be included as well. Furthermore, server devices 202 may organize the received data into web page or web application representations. Such a representation may take the form of a markup language, such as HTML, XML, JSON, or some other standardized or proprietary format. Moreover, server devices 202 may have the capability of executing various types of computerized scripting languages, such as but not limited to Perl, Python, PHP Hypertext Preprocessor (PHP), Active Server Pages (ASP), JAVASCRIPT®, and so on. Computer program code written in these languages may facilitate the provision of web pages to client devices, as well as client device interaction with the web pages. Alternatively or additionally, JAVA® may be used to facilitate generation of web pages and/or to provide web application functionality.

### III. Disease Prediction from Large Populations and High-Dimensional Features

Predicting disease risk from DNA presents an unprecedented emerging challenge, as biobanks approach population scale sizes of N>1,000,000 individuals with high-dimensional features of L>100,000 genotypes. Current methods, often linear and reliant on summary statistics, fail to capture complex genetic interactions and discard valuable individual-level information.

The embodiments herein introduce PRSformer, a scalable deep learning architecture designed for end-to-end, multitask disease prediction directly from million-scale individual genotypes. PRSformer employs neighborhood attention, achieving linear O(L) complexity per layer, making transformers tractable for genome-scale inputs. Particularly, PRSformer utilizes a stacking of these efficient attention layers, progressively increasing the effective receptive field to model local dependencies (e.g., within LD blocks) before integrating information across wider genomic regions. This design, tailored for genomics but potentially applicable more generally, allows PRSformer to learn complex, potentially non-linear and long range interactions directly from raw genotypes.

PRSformer's effectiveness is demonstrated using a unique large private cohort (N≈5,000,000) for predicting autoimmune diseases using L≈140,000 variants. PRSformer significantly outperforms highly optimized linear models trained on the same individual-level data and state-of-the-art summary-statistic-based methods (LDPred2) derived from the same cohort, quantifying the benefits of non-linear modeling and multitask learning at scale. Furthermore, experiments reveal that the advantage of non-linearity emerges primarily at large sample sizes (N>1,000, 000), and a multi-ancestry trained PRSformer shows improved generalization to diverse populations.

Specifically, PRSformer was trained and validated in a multitask setting across autoimmune and inflammatory conditions. This disease set provided a testbed for multitask learning due to autoimmune diseases frequently exhibiting shared inflammatory pathophysiology and overlapping genetic factors. The multitask formulation allows testing of PRSformer's ability to exploit shared genetic associations while learning disease-specific patterns, aiming to enhance predictive performance through a shared representation.

Rigorous empirical validation is provided using data from a unique large private cohort (N≈3,800,000 European-ancestry individuals) for training, validation, and evaluation across D=18 autoimmune diseases using L≈140,000 variants. The scale of this cohort significantly exceeds current public biobanks, providing a real-world testbed for methods designed to handle genomic data of the million-scale magnitude.

Comparisons are conducted against: (i) highly optimized linear models (regularized logistic regression with learnable embeddings) trained on the exact same individual-level genotype data, isolating the benefit of PRSformer's non-linear architecture; and (ii) state-of-the-art summary-statistic-based PRS methods (e.g., LDPred2) derived from the same cohort, enabling a direct comparison between end-to-end and summary-statistic-based approaches.

Experimental data demonstrates statistically significant performance gains for PRSformer. Performance across varying training sample sizes can be analyzed, revealing that the benefits of non-linearity become pronounced at large scales (N>1,000,000). In addition, multitask training across diseases consistently outperforms the more common single-task paradigm, highlighting the advantage of learning shared genetic representations across related traits. Additionally, improved cross-ancestry generalization is demonstrated by training PRSformer on an additional multi-ancestry cohort.

Among other technical advantages, PRSformer provides deep learning techniques for genomics at population scale, and is the first efficient multitask transformer architecture applied to population-scale data with hundred-thousand-plus features. Further, rigorous validation showing significant predictive improvements over optimized linear models and state-of-the-art summary-statistic methods on the same data, directly quantifying the advantage of PRSformer's end-to-end, non-linear approach, particularly evident at large sample sizes. Moreover, PRSformer provides improved cross-ancestry generalization. Training PRSformer on aggregated multi-ancestry data, including approximately an additional 1.1 million non-European individuals, enhances prediction accuracy in held-out non-European individuals compared to a European-only trained model.

Thus, PRSformer establishes a beneficial new framework for deep learning in population-scale genomics and offers insights into modeling ultra-long genomic sequences. And PRSformer does all of this in a computationally efficient fashion, with the aforementioned linear O(L) complexity per layer as opposed to O(L²) computational complexity of standard self-attention transformer architectures. Therefore, PRSformer uses dramatically fewer computational resources (e.g., processor, memory, and power capacity) to achieve superior results.

Traditional complex trait prediction relies heavily on PRS derived from GWAS summary statistics. Early methods often involved simple thresholding and summing of SNP effects. More recent Bayesian approaches, such as LDpred2 and PRS-CS, explicitly model LD patterns and utilize shrinkage priors to improve predictive accuracy. These methods represent the current state-of-the-art for prediction from summary statistics.

However, PRS methods based on summary statistics are fundamentally limited in several ways. First, by discarding individual-level genotype and haplotype information, these methods cannot capture LD structure and instead rely on LD estimates imputed from external reference panels, which can introduce biases due to population mismatches. Second they cannot capture variant-variant interactions such as epistasis as they are restricted to using marginal variant effects. Third, the use of precomputed summary statistics constrains these models to largely linear architectures, precluding the discovery of complex multi-locus or hierarchical genetic patterns. These limitations cap predictive performance, particularly as biobank-scale datasets grow large enough to enable the detection of more subtle and nonlinear genetic effects.

Deep learning has been successfully applied to various supervised genomic prediction tasks. Much work has focused on modeling sequence-level information (DNA base pairs) to predict molecular phenotypes like transcription factor binding, chromatin accessibility, or gene expression. These approaches have predominantly utilized convolutional neural networks (CNNs) or transformers incorporating CNN-style tokenization, which are well-suited to capturing biologically meaningful motifs and local patterns at base-pair resolution. However, this paradigm is less applicable when modeling the influence of genetic variants (e.g., SNPs) on complex traits, as causal variants can be spread across the genome and may interact over long distances, often without strong local sequence motifs defining their impact.

The application of deep learning to predict complex traits (like disease status) directly from individual-level genotype data (i.e., SNP arrays) remains relatively underexplored, particularly at the population scale addressed herein. This is largely due to the challenges of ultra-high dimensionality (L) and, until recently, the limited statistical power of publicly available cohorts with both individual-level genotypes and phenotypes (N). Prior work in this specific domain has often relied on: (i) tree ensemble models such as gradient boosting or simple neural networks trained on reduced feature sets (e.g., using LASSO feature selection); (ii) smaller cohorts where complex interactions are difficult to detect; and (iii) models operating on precomputed PRS or summary statistics rather than raw genotype data.

Applying standard transformers to genome-scale data (L>100,000) is computationally prohibitive due to the O(L²) complexity of self-attention. A wide range of efficient attention mechanisms have been proposed to address this limitation, including sparse attention patterns (e.g., Longformer, BigBird), low-rank approximations (e.g., Linformer), and kernel-based methods (e.g., Performer). Other architectures exploit locality through sliding windows or blockwise mechanisms (e.g., Swin Transformer) to reduce complexity while capturing local dependencies. The embodiments herein employ neighborhood attention, a variant of self-attention in which each query attends only to a fixed-size local window of neighboring tokens, rather than the full sequence. Doing so aligns well with the block-like correlation structure of genomic data driven LD. By limiting attention to a neighborhood of size k<<L, NA reduces both computational and memory complexity to O(L·k), achieving linear scaling in sequence length. An optimized GPU implementation provided by the NATTEN library is used, which supports scalable training on long sequences while maintaining the expressiveness of content-based attention.

### IV. Implementation Details

A goal of PRSformer is to predict susceptibility to multiple (D=18 in these examples) autoimmune diseases from individual-level genotypes. Formally, given a dataset of N individuals, the input for individual i is their genotype profile xᵢ∈{0, 1, 2, UNKN}^{L}, representing dosages of L pre-selected genetic variants, where UNKN indicates missing data. The target output is a vector yᵢ∈{0, 1, UNKN}^{D}, representing the binary status (case/control) for D diseases, where UNKN indicates unrecorded status. Doing so may involve learning a multitask function ƒ: Z^{L}→[0, 1]^{D} that predicts the probability of each disease ŷⁱ=ƒ(xᵢ). The primary challenges lie in the ultra-high dimensionality (L≈140,000 in this work), the benefit of capturing potentially non-linear and long-range interactions, and leveraging the statistical power of population-scale datasets (N≈5,000,000 total used in this study). A transformer based architecture is adapted for this task, employing efficient attention mechanisms for scalability and multi-task learning for joint prediction across diseases.

### A. Architecture

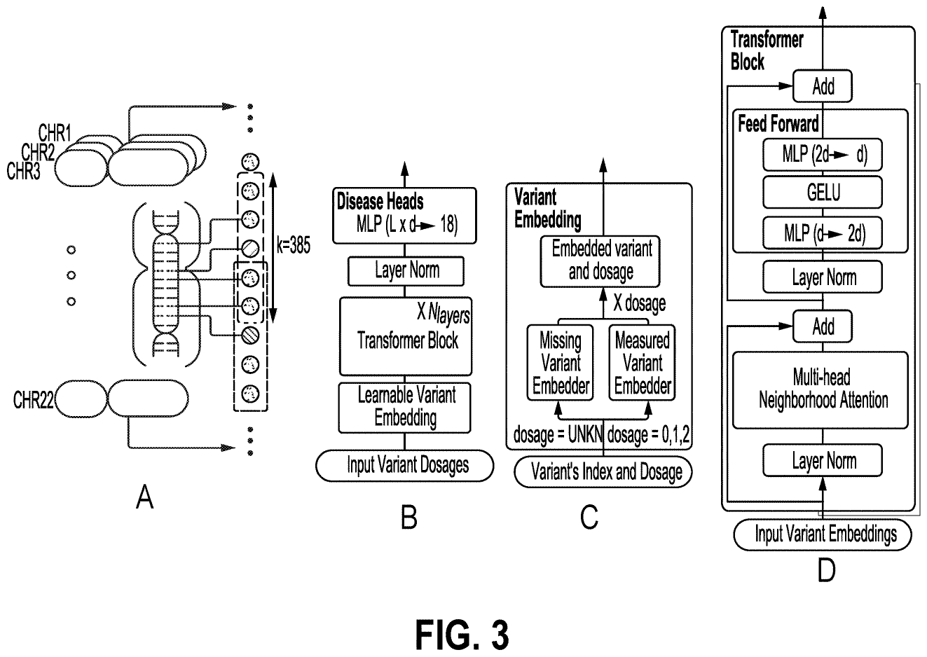

FIG. 3 depicts the PRSformer architecture. Part (A) represents a preselected set of variants across at least 22 chromosomes (e.g., the autosomes), sorted by genomic position, that allows each query variant to attend within a local block (k=385) via neighborhood attention. Part (B) represents the model architecture with input, transformer blocks, and output layers. Part (C) represents a variant embedding layer, which encodes each variant and its corresponding dosage (0, 1, 2, or UNKN) into a 64-dimensional representation. Part (D) represents a transformer block with pre-layer normalization, neighborhood attention, and Gaussian error linear unit (GELU) activation.

PRSformer adapts the transformer architecture for disease prediction from ultra-long (L≈140,000) individual-level genotype sequences using a number of design features, including neighborhood attention and genome-ordered input without explicit positional encodings.

Standard O(L²) self-attention is computationally infeasible. It is replaced with neighborhood attention (NA), restricting each query token's attention to a symmetric local window of size k. This reduces complexity to O(L·k), enabling efficient processing of the L=137, 245 input variants used in these experiments. Also, k=385 is chosen via hyperparameter tuning (see below), which conceptually aligns with capturing dependencies within local LD blocks (see FIG. 3 Part (A)) and corresponds to roughly≈100 kilobases along the genome.

Input variants are sorted by their genomic position (e.g., chromosome 1 to chromosome 22), and this fixed order is used for all individuals. Standard positional encodings (e.g., sinusoidal or learned absolute) can be omitted. The fixed genomic order provides implicit relative positional information that NA inherently considers within its local attention windows.

The overall data flow of PRSformer proceeds in accordance with FIG. 3 Part (B).

The learnable variant embedding layer (described more fully in FIG. 3 Part (C)) receives the input variant dosages. Each variant in this input sequence of variant dosages is mapped to a d_{model}-dimensional vector. For each variant j, an observed dosage xᵢⱼ∈{0, 1, 2} is represented as Eⱼ·xᵢⱼ using a learned variant-specific embedding Eⱼ. A missing genotype (xᵢⱼ=UNKN) is represented by a separate learned variant-specific embedding Mⱼ. This allows the model to distinguish missingness from observed genotypes distinctly for each variant. A value of d_{model}=64 was used.

The transformer blocks (described more fully in FIG. 3 Part (D)) receive the output of the learnable variant embedding layer. The embedded sequence is processed by N_{layers}=2 transformer blocks. Each block applies pre-layer normalization followed by multi-head NA (N_{heads}=4) and a feed-forward network with GELU activation. The choice of N_{layers}=2 was based on validation performance, where deeper models did not show significant improvement for this task (see below for a further discussion), suggesting two stacked NA layers provide a sufficient receptive field to capture interactions between adjacent LD blocks.

The final sequence representation from the last block is passed to a fully connected layer generating D=18 independent disease likelihood predictions.

Architectural hyperparameters (d_{model}, N_{heads}, N_{layers}, and NA window size k) were obtained based on validation set performance (see below for a further discussion). However, other hyperparameters can be used and/or these hyperparameters can be obtained in a different way.

### B. Datasets

Data from a large, private biobank consisting of individuals who consented to participate in research was used. Starting from an internal GWAS data lock timestamped August 2021 (used to prevent information leakage), individuals genotyped on the same platform were identified. Individuals included had recorded phenotypes (e.g., self-reported status) for at least one of the D=18 autoimmune diseases considered. Individuals under age 10 who did not have a case diagnosis were excluded. This resulted in a training set of Nₜᵣₐᵢₙ=3,838, 549 individuals of European ancestry (ancestry determined via an internal classifier).

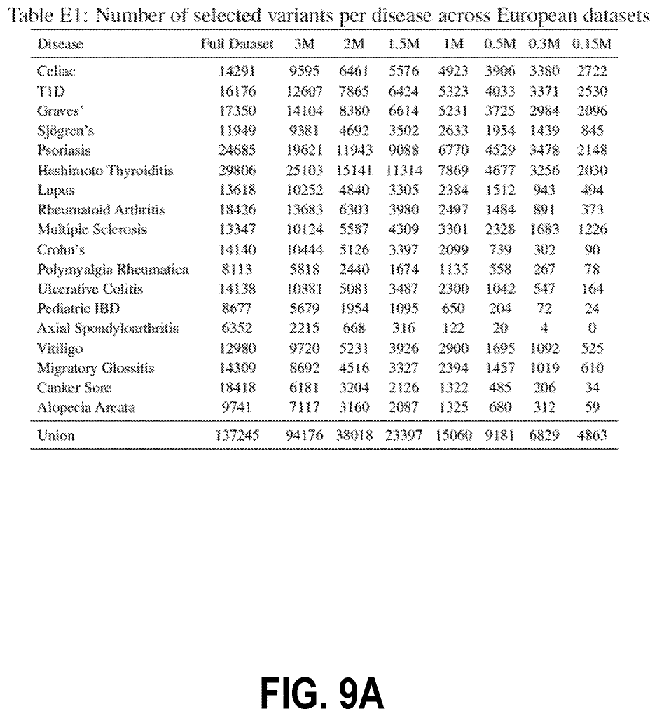

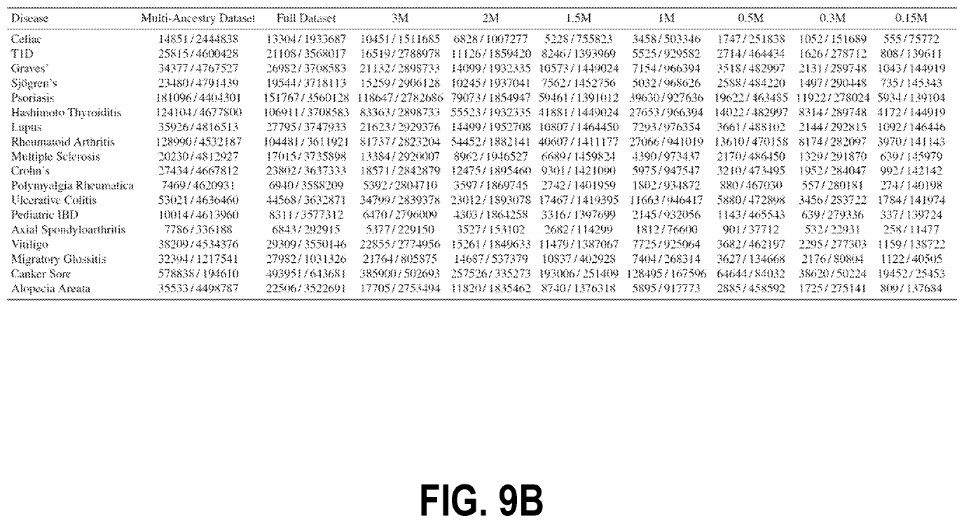

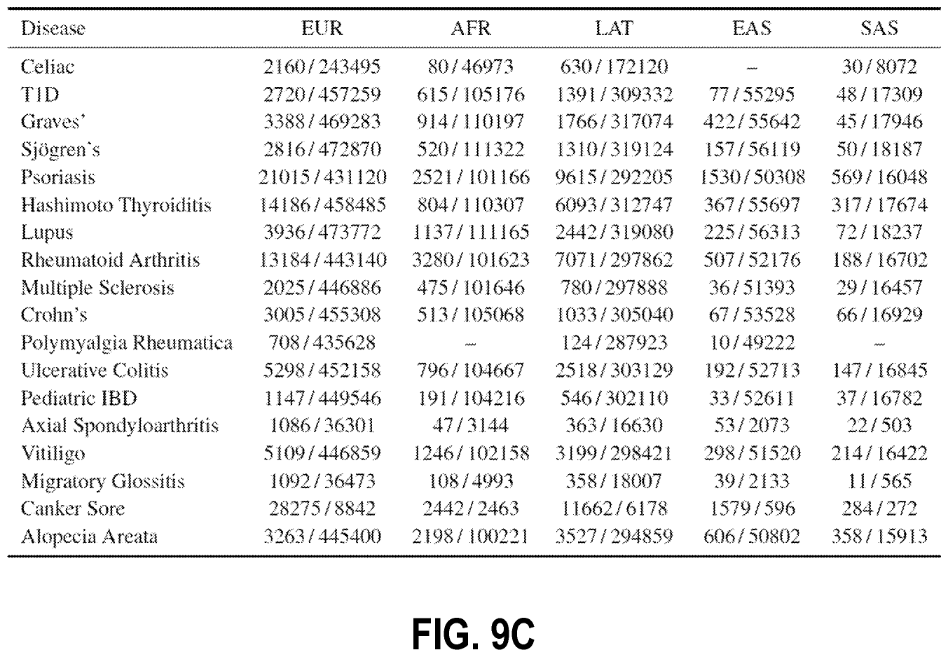

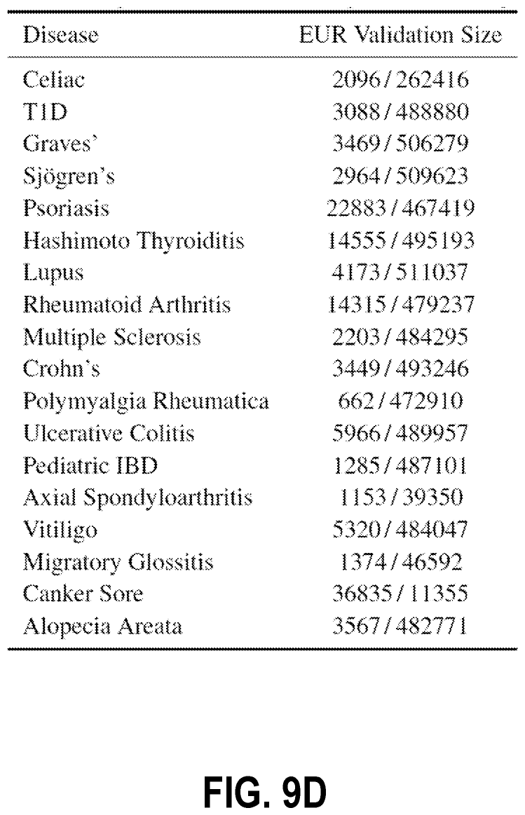

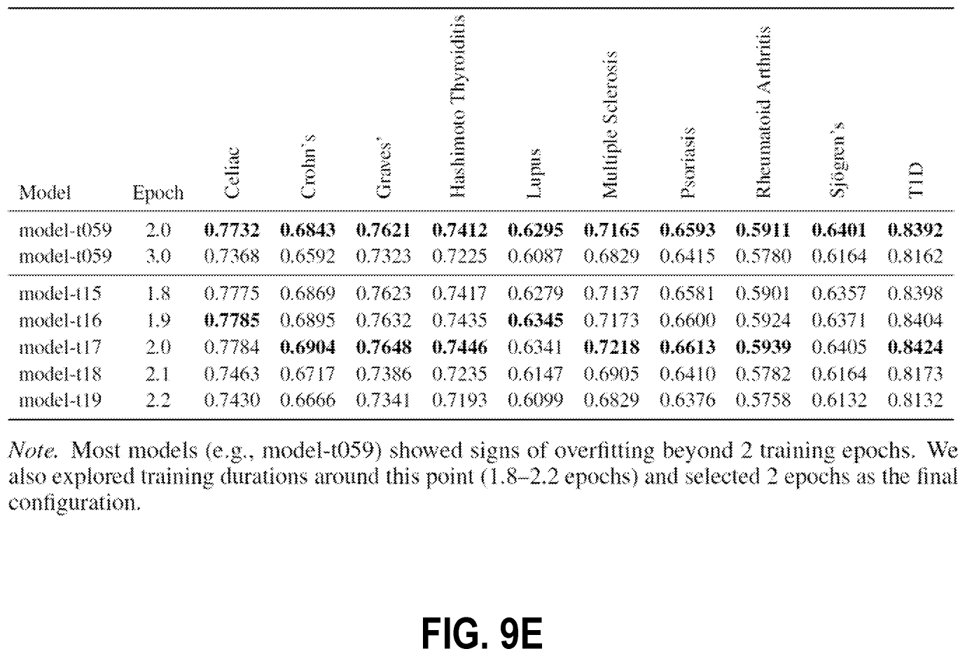

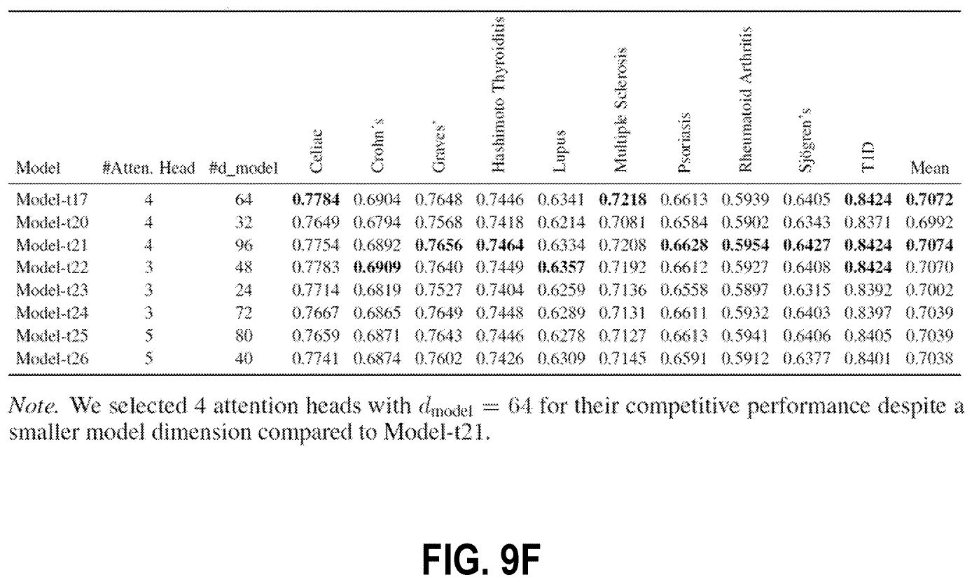

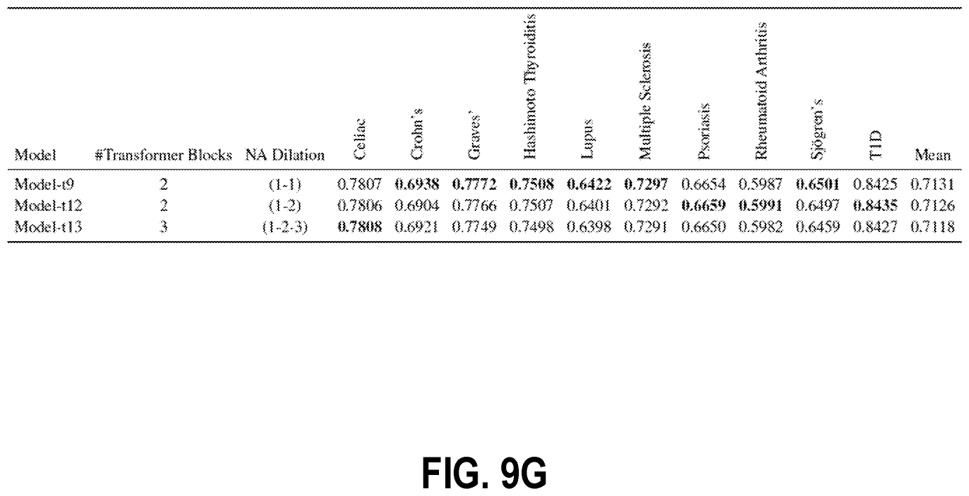

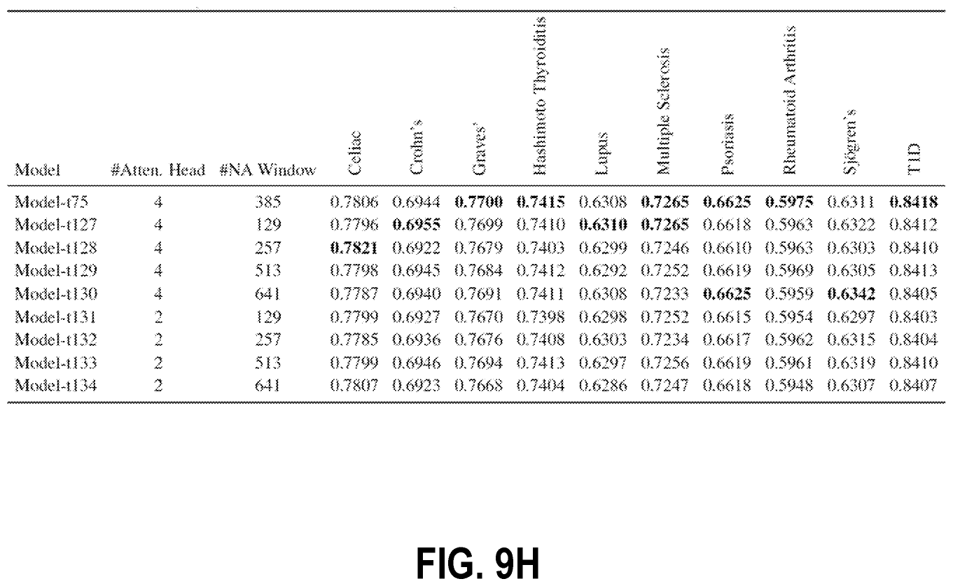

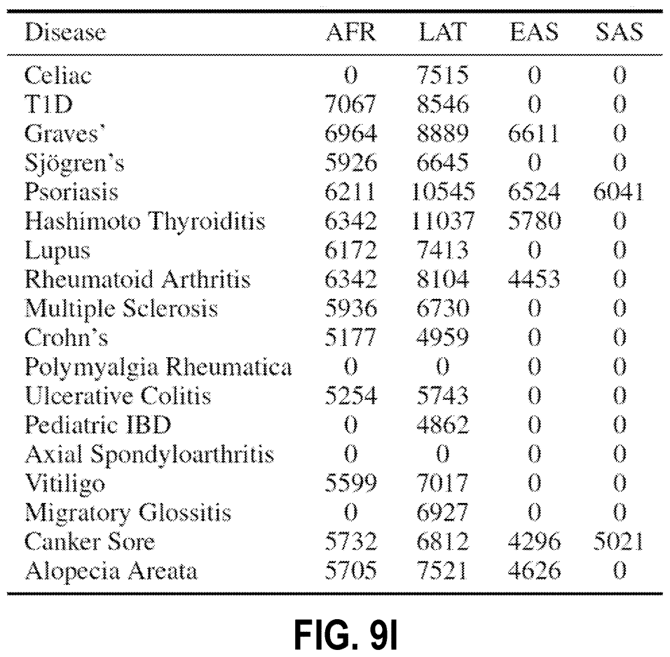

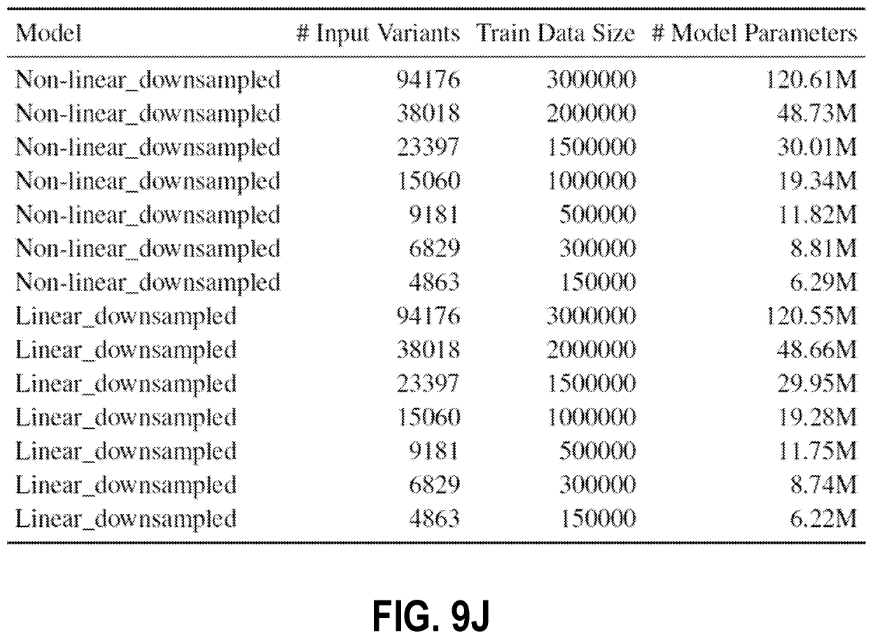

Temporally distinct validation and test sets were constructed using individuals who enrolled and consented after the August 2021 data lock date and up to December 2024, applying the identical filtering criteria. The validation dataset was used for hyperparameter tuning, while the test dataset was used to report final performance metrics. This yielded Nᵥₐₗ=525, 448 and Nₜₑₛₜ=494, 265 individuals. Total individual counts were N=4, 858, 302. An additional N≈1,100,000 non-European individuals were selected for cross-ancestry modeling. Case/control counts per disease and further details regarding cohort construction (including differences from the subset used for GWAS) are provided in Appendix A and FIGS. 9B-9D.

### C. Variant Selection

To define the input feature space (L), variants associated with at least one of the D=18 diseases were selected based on internal GWAS summary statistics (European ancestry cohort, computed prior to the August 2021 data lock to prevent information leakage into model training). For each disease, variants passing quality control, located on autosomal chromosomes, had a genotyping rate≥0.95, minor allele frequency>0.001, and exhibited nominal association with the disease (GWAS p-value<1×10⁻²). The final PRSformer input set was the union across all 18 diseases, resulting in L=137,245 variants. Further details on GWAS procedures and per-disease variant counts are in Appendix A and FIG. 9A.

### D. Training

Given the training data D={(xᵢ, yᵢ)}; ᵢ^{N,train}, PRSformer was trained by minimizing the following loss, summed over individuals and their available (non-UNKN) disease labels t∈T(i):

$$ℒ = - {\sum }_{i = 1}^{{N}_{train}} {\sum }_{t ∈ T ( i )} [ {y}_{i , t} log ( {\hat{y}}_{i , t} ) + ( 1 - {y}_{i , t} ) log ( 1 - {\hat{y}}_{i , t} ) ]$$

Where T(i) denotes the set of recorded disease statuses for individual i. This summation (rather than averaging) naturally weights the contribution of each disease task by the number of available labels, implicitly handling imbalance across diseases. The AdamW optimizer (β₁=0.9, β₂=0.999, weight decay=0.05) was used with an initial learning rate of 5×10⁻⁴, decreased via a Cosine Annealing scheduler. Training was performed efficiently on this large-scale dataset for 2 epochs consisting of ≈120,000 gradient updates in an effective batch size of 64 across four NVIDIA A100 GPUs, leveraging distributed data parallel and mixed-precision (FP16) training. Hyperparameters, including architecture choices (N_{layers}, d_{model}, N_{heads}, k), were selected based on area under the receiver operating characteristic (AUROC) on the validation set after extensive searches (e.g., see FIGS. 9A-9J).

### E. Baseline Models

To comprehensively evaluate PRSformer and validate the utility of end-to-end non-linear modeling on large-scale individual-level genotypes, three rigorous baselines were established. These baselines are specifically designed to: (1) compare against the current state-of-the-art using conventional summary-statistic inputs (LDPred2), (2) benchmark against an enhanced version of this state-of-the-art (Stacked LDPred2), and (3) isolate the specific performance gains attributable to PRSformer's non-linear transformer architecture via a carefully matched linear counterpart.

LDPred2 is a state-of-the-art summary-statistic method selected due to its strong empirical performance and widespread adoption in the field. To provide a direct comparison, LDPred2 was configured as follows. Matched data source: LDPred2 was applied to GWAS summary statistics derived from the same European training data lock used for PRSformer's data and variant selection. Cohort-specific LD: An LD reference panel from the research cohort was used. Standard QC: Input variants (~445,000 per disease) passed standard GWAS quality control and LDPred2-specific filtering. Tuning: LDPred2 hyperparameters (p, h2) were extensively tuned (up to 100 models per disease) by maximizing AUROC on the same validation set used for PRSformer (see Appendix B).

Stacked LDPred2 is an enhanced summary-statistic baseline. To create a stronger summary statistic baseline, the converged LDPred2 models were assembled from the hyperparameter search using elastic-net regression trained via cross-validation on the validation set (see Appendix B, below). Since Stacked LDPred2 uses the validation set for training ensemble weights, it is compared against PRSformer+, which is the final PRSformer model retrained on the combined training and validation datasets, providing parity in total data usage (see FIGS. 8B-1 and 8B-2).

The linear model uses a direct architectural ablation that isolates the contribution of PRSformer's non-linear transformer architecture. It mirrors PRSformer except for omitting the transformer blocks. The linear model (i) uses the exact same L≈140,000 input variants and individual-level train/validation/test splits, (ii) employs the same multitask framework (D=18), loss function, AdamW optimizer, and training schedule, and (iii) uses the same embedding layer (see FIG. 3 Part (C)) for dosages and missingness. The input embeddings are fed directly to the final linear output layer, bypassing the transformer blocks (see FIG. 3 Part (D)). This architectural change provides a multitask linear model on the same large-scale individual data, allowing direct assessment of the performance gain from PRSformer's non-linear processing.

### F. Cross-Ancestry Experiments

To assess generalization, PRSformer-ME (Multi-Ethnic) was developed. Ancestry-specific GWAS (AFR, LAT, EAS, SAS; determined by internal classifier) was performed using the same August 2021 data lock and variant selection criteria (see above) where sample sizes permitted (see FIG. 9I). An expanded input set (L=251,538 variants) was defined as the union of selected variants across all available disease-ancestry pairs (including Europeans). A multi-ancestry training set (~5,000,000 total individuals) was constructed by combining the European training set (see above) with N=1,136,746 non-European individuals meeting the same filtering criteria. PRSformer-ME was trained on this combined dataset using the same architecture and hyperparameters as the European-only PRSformer, without additional ancestry-specific tuning. Evaluation was performed on a combined test set including the European test set and held-out non-European individuals processed identically (see FIG. 9C).

### V. Experimental Results

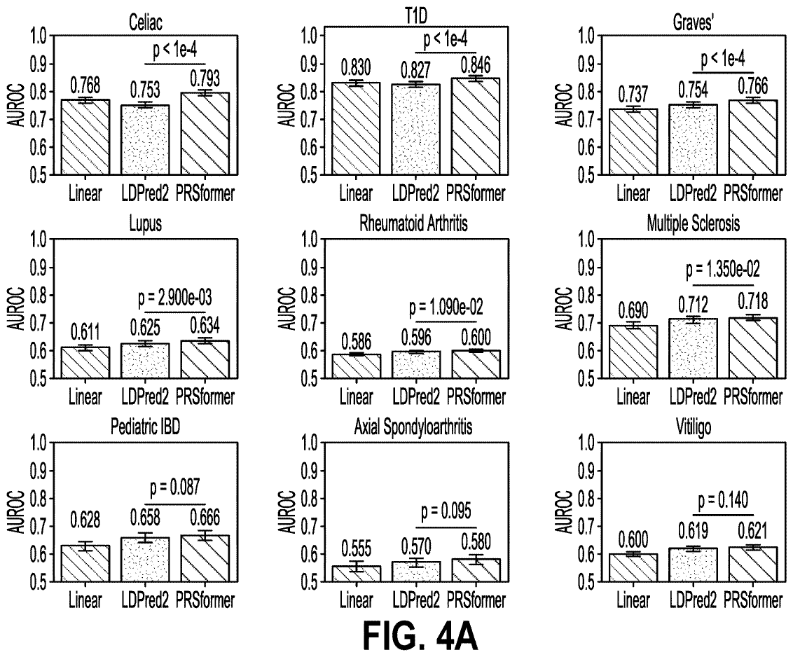

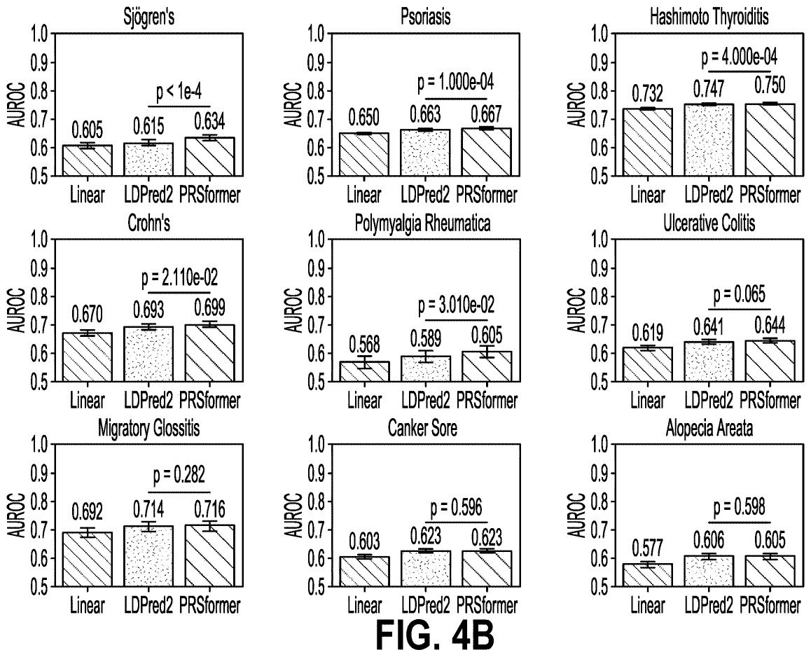

PRSformer was benchmarked against the highly optimized linear model and the state-of-the-art summary-statistic method, LDPred2, on the European test set (Nₜₑₛₜ≈494,000). As shown in FIGS. 4A and 4B, PRSformer consistently achieves higher AUROC scores than its linear counterpart across nearly all 18 autoimmune diseases. This comparison, using identical data and training setups except for the transformer blocks, directly quantifies the predictive benefit derived from PRSformer's non-linear architecture.

PRSformer also significantly outperforms LDPred2 (using summary statistics derived from the same cohort) on 16 out of 18 diseases, with 11 differences being statistically significant (p<0.05, one-sided paired bootstrap test). This demonstrates the advantage of end-to-end modeling on individual-level data compared to state-of-the-art methods relying on summary statistics. Consistent improvements were also observed using area under the precision-recall curve (AUPRC) (see FIGS. 8A-1, 8A-2, and 8A-3) and when comparing against an enhanced Stacked LDPred2 baseline (see FIGS. 8B-1 and 8B-2), confirming the robustness of PRSformer's advantage.

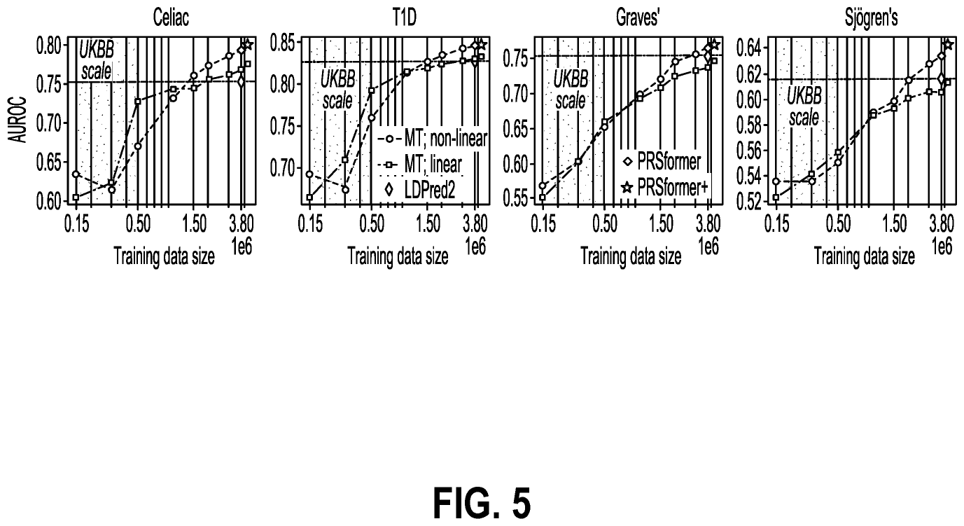

To understand when the non-linear modeling capabilities of PRSformer become advantageous, its performance was compared against the linear baseline across varying training dataset sizes (downsampling the N≈3,800,000 training set). FIG. 5 reveals that, at smaller sample sizes, comparable to current large public cohorts like UK Biobank (up to N≈1,000,000), the performance of PRSformer is similar to the simpler linear model. However, as the training size exceeds one million individuals, a clear advantage for the non-linear PRSformer emerges and progressively widens. This trend holds across multiple diseases (see FIGS. 8C-1 and 8C-2) and persists even when using appropriately subsetted variant sets for smaller scales (see Appendix C, FIGS. 8D-1 and 8D-2). This finding strongly suggests that capturing complex, non-linear genetic effects requires the statistical power afforded by massive, million-plus sample sizes, highlighting the importance of scalable architectures like PRSformer for leveraging next-generation biobanks.

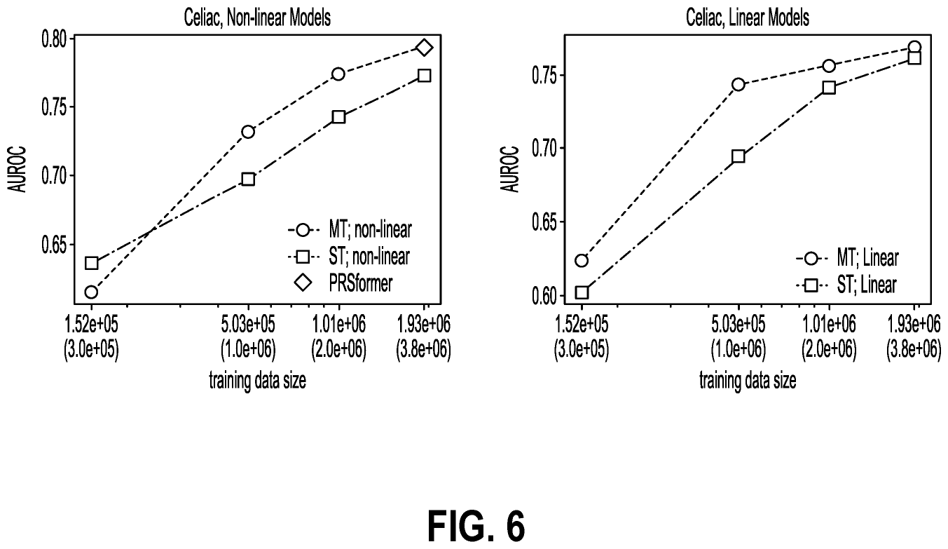

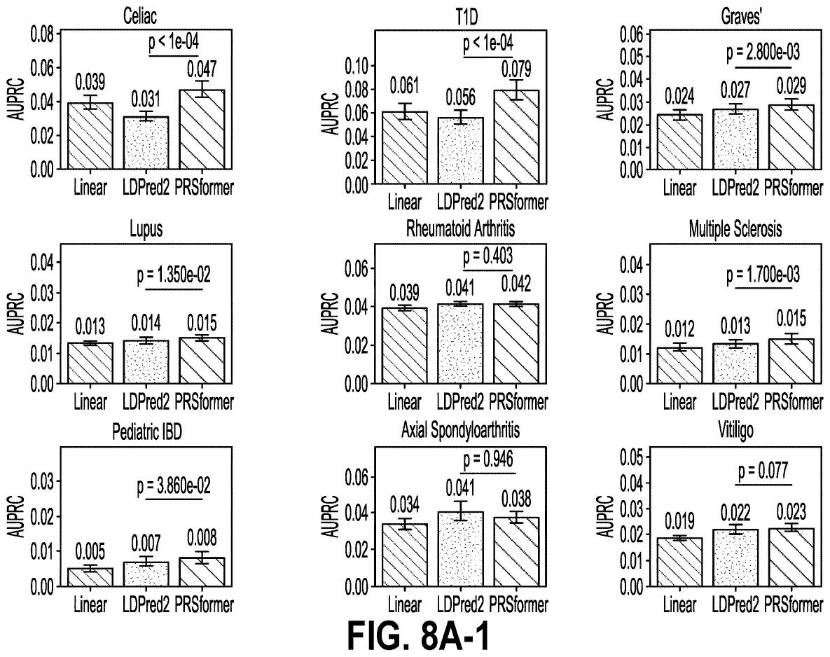

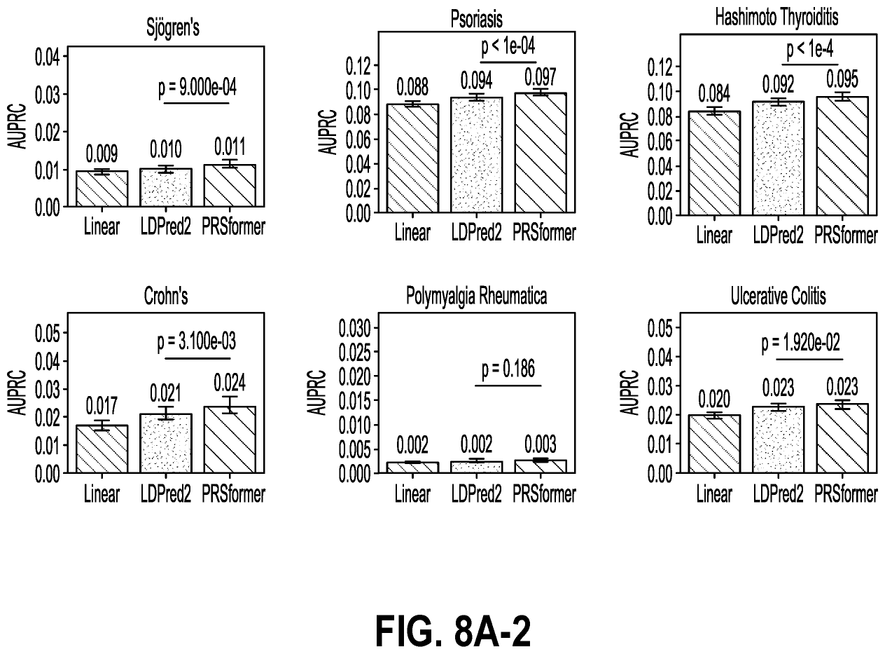

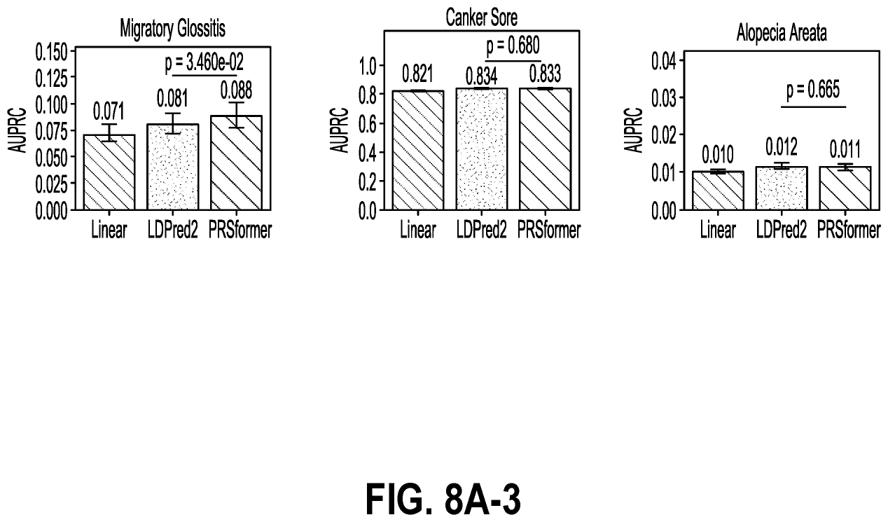

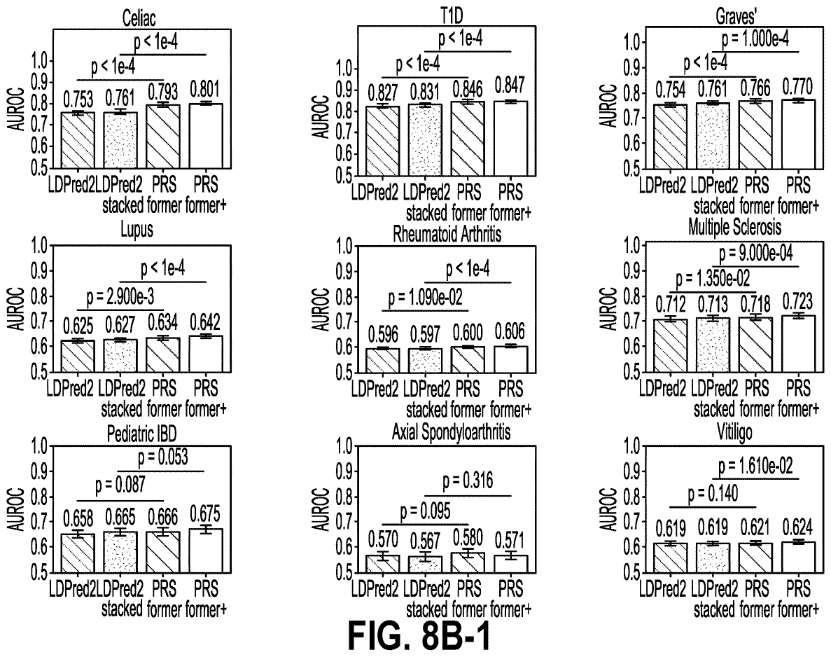

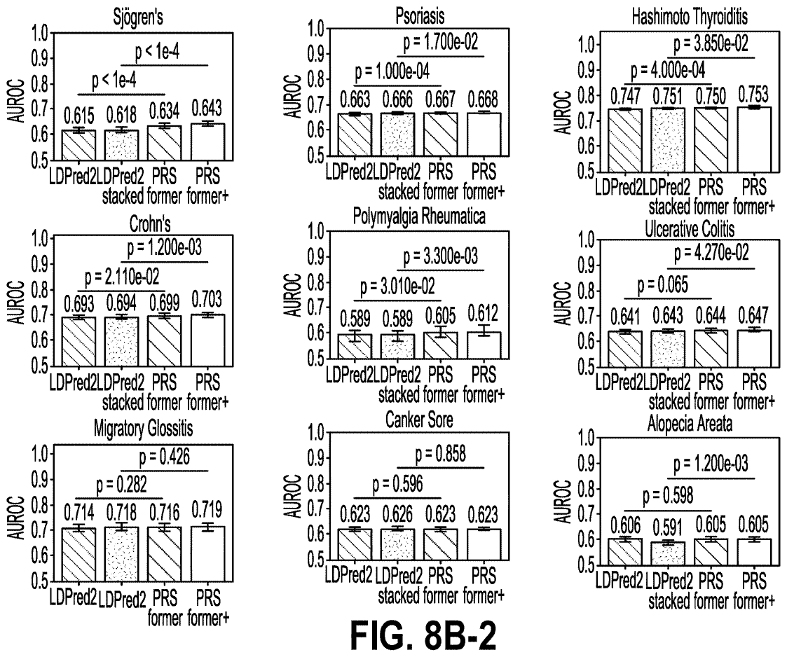

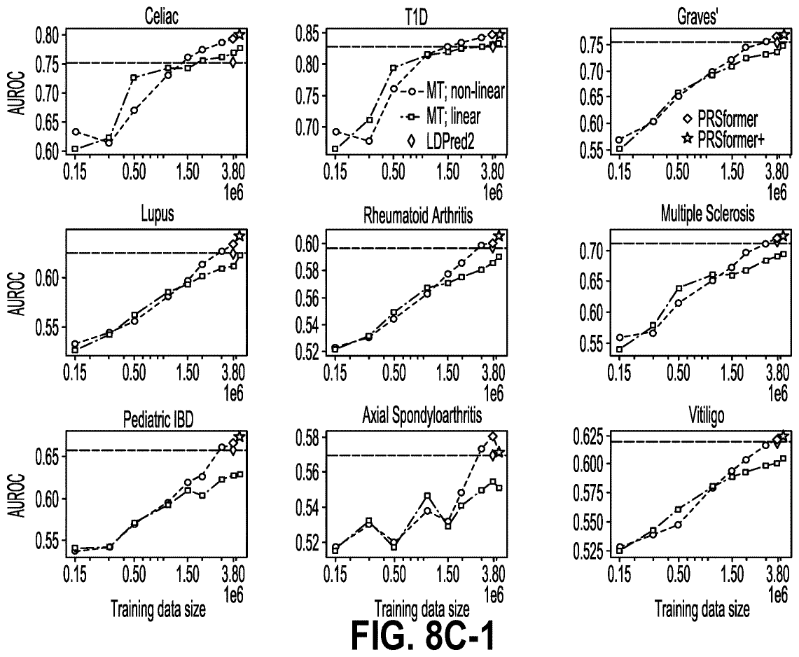

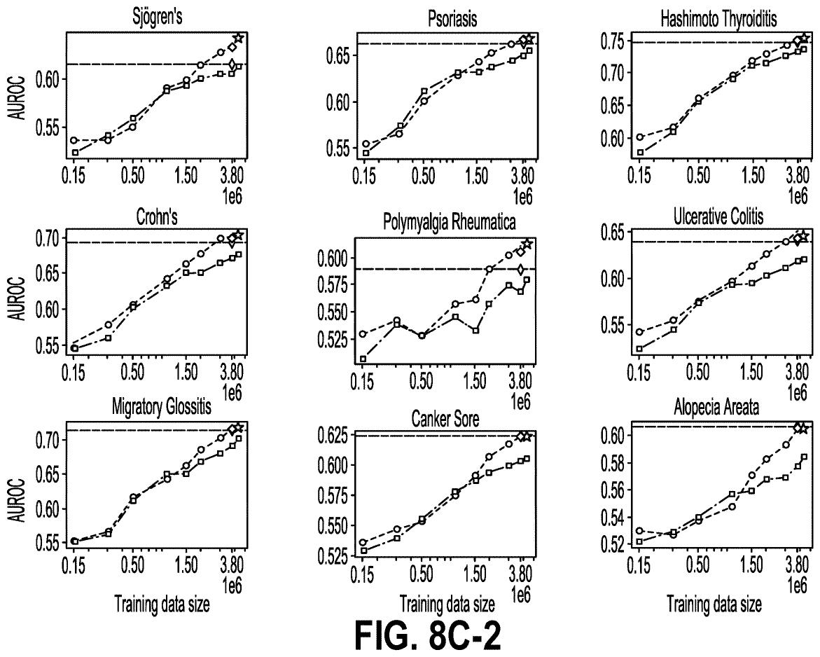

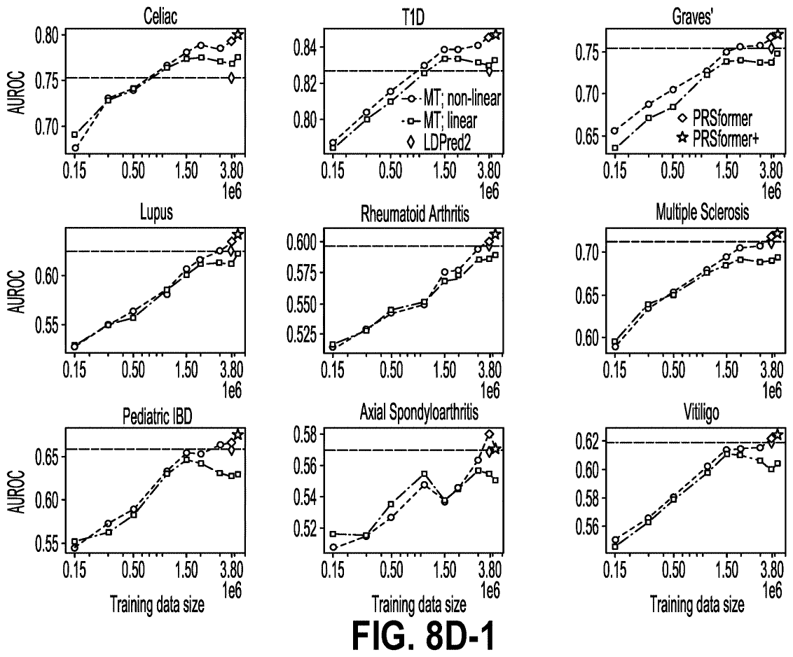

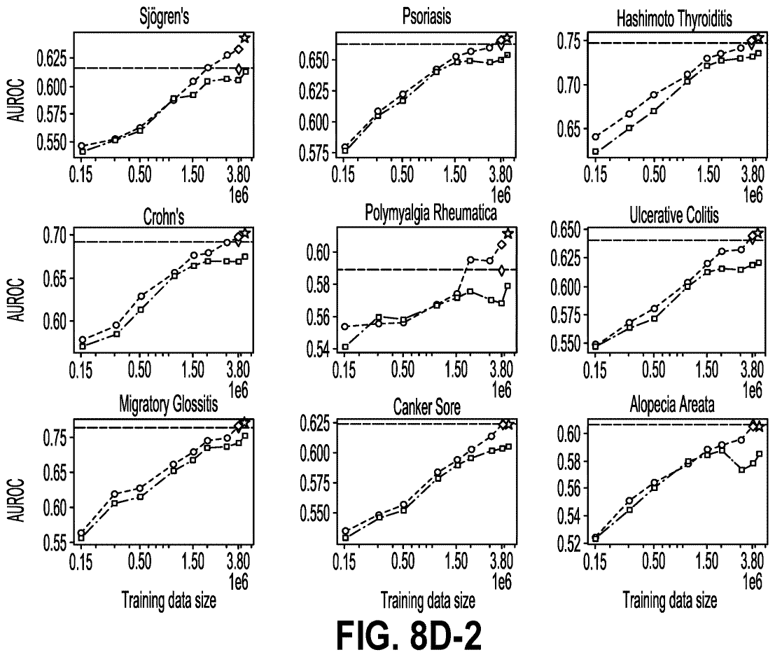

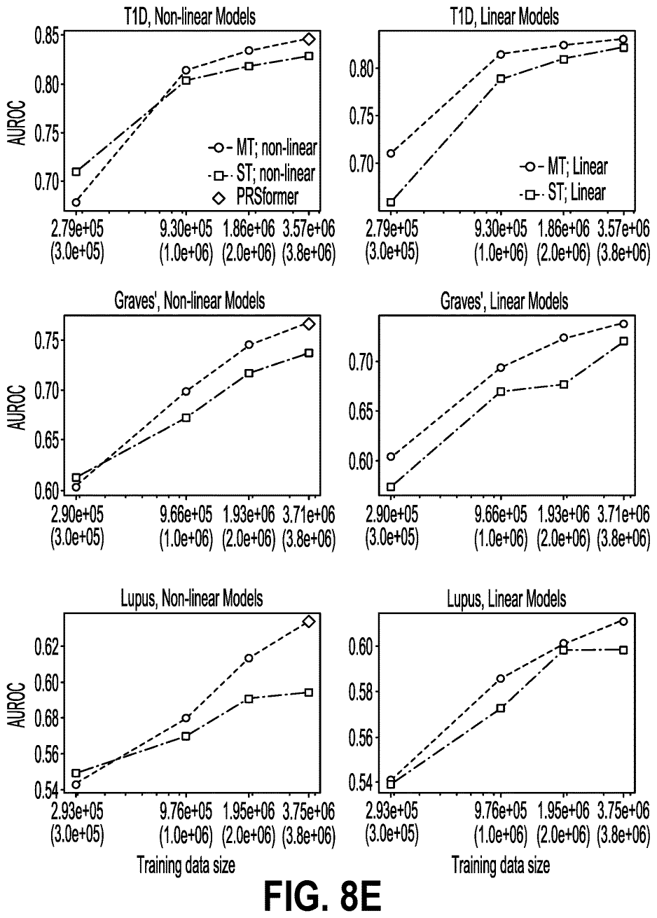

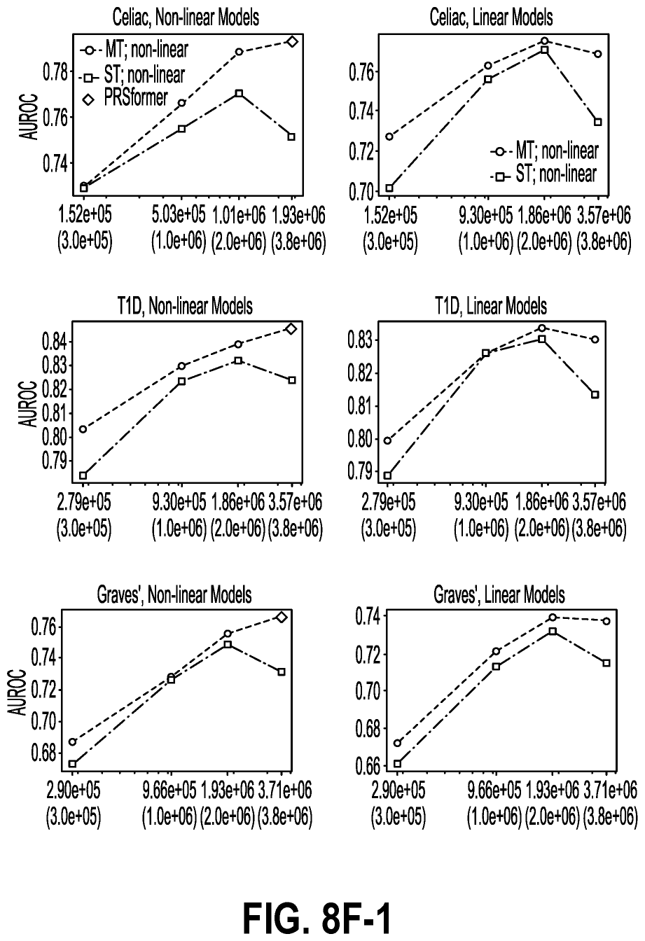

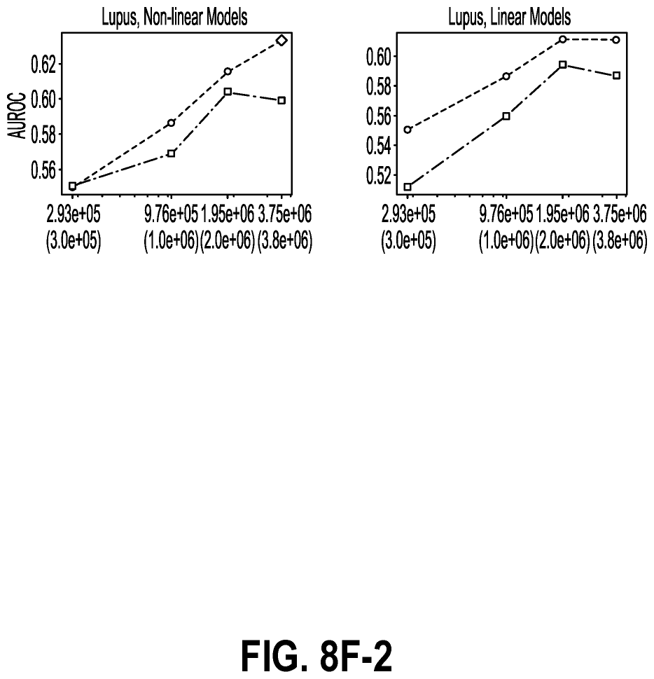

The benefit of PRSformer's multitask design was investigated by comparing it against single-task (ST) models trained independently for each disease. FIG. 6 shows that multitask (MT) training consistently yields superior AUROC compared to single-task training for both PRSformer and the linear baseline, across different data scales (see FIG. 8E for other diseases). This improvement was robust even when single-task models used disease-specific optimized variant sets (see FIGS. 8F-1 and 8F-2). The gain stems from leveraging shared information across related autoimmune diseases allowing shared model components (variant embeddings and transformer blocks in PRSformer, and variant embedding in the linear baseline) to be optimized more effectively. By training these shared layers across multiple related diseases, the model can capture generalizable representations that enhance performance beyond what is achievable with isolated, single-task training.

Recognizing the need for equitable genomic prediction, PRSformer-ME was trained on a combined multi-ancestry cohort (~5,000,000 individuals, including ~1,100,000 non-Europeans) using an expanded variant set (L≈252,000, see above). Its performance was evaluated on a held-out test set containing individuals from European (EUR), African American (AFR), Latino (LAT), East Asian (EAS), and South Asian (SAS) ancestries, comparing it to the original PRSformer trained only on Europeans.

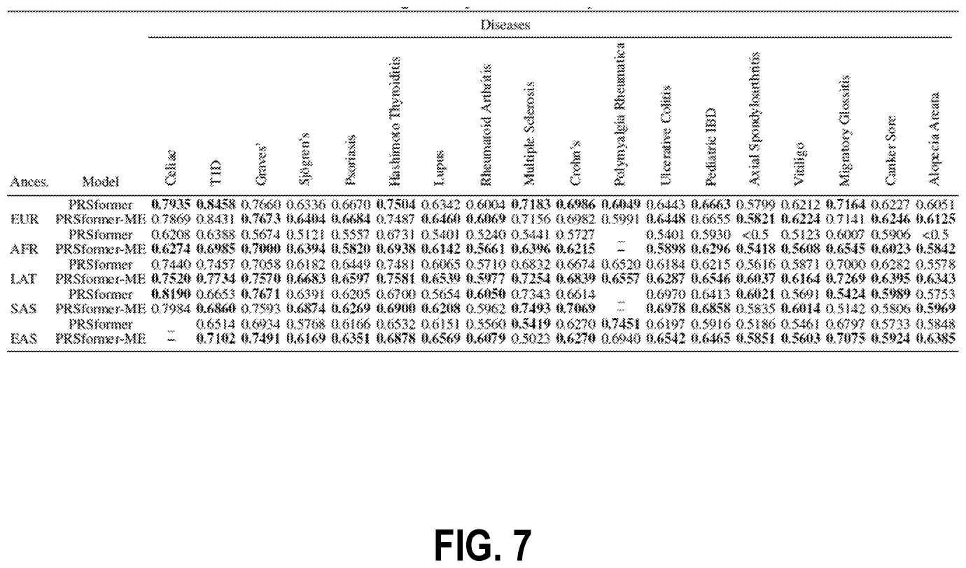

As summarized in FIG. 7, PRSformer-ME demonstrates significantly improved generalization to non-European populations. It achieves substantially higher AUROC scores across most diseases in AFR, LAT, EAS, and SAS individuals compared to the EUR-only model. Importantly, this gain in non-European populations is achieved with minimal to no degradation in performance on European individuals. These results indicate that training on diverse, aggregated individual-level data allows PRSformer-ME to capture both shared and ancestry-specific genetic signals, leading to more accurate and potentially more equitable predictions across populations compared to models trained on a single ancestry group.

### VI. Example Technical Improvements

These embodiments provide a technical solution to a technical problem. One technical problem being solved is the computational intractability of applying high-capacity sequence modeling architectures to ultra-high-dimensional genomic data. In practice, this is problematic because standard self-attention mechanisms of transformers exhibit quadratic O(L²) complexity (where L represents the number of genetic variants), which renders them computationally infeasible for genome-scale inputs where the number of genetic variants approaches or exceeds 100,000. This creates a scalability bottleneck that prevents the use of sophisticated deep learning models on population-scale biobanks, effectively capping the predictive power of genomic models as datasets grow.

In other techniques, disease risk prediction primarily relies on linear PRS derived from GWAS summary statistics. However, these techniques do not capture complex, non-linear genetic interactions and discard valuable individual-level haplotype information. Moreover, other approaches rely on subjective decisions in choosing external reference panels for LD estimation, which leads to varying outcomes from instance to instance due to population mismatches. Thus, other techniques did little if anything to address the need for an end-to-end, scalable architecture capable of learning directly from raw, million-scale individual genotypes.

The embodiments herein overcome these limitations by incorporating a neighborhood attention mechanism that restricts computations to local genomic windows, reducing complexity to linear O(L) per layer. In this manner, joint prediction across multiple related diseases (multitask learning) can be accomplished in a more accurate and robust fashion using ultra-long sequences. This results in several advantages. First, the processor and memory efficiency of the neighborhood attention architecture allows for the training of deep models on sequences of L≈140,000 to L≈252,000 variants, which was previously computationally prohibitive. Second, the stacking of efficient attention layers allows the model to capture local dependencies within LD blocks before integrating information across wider genomic regions. Third, the architecture demonstrates improved cross-ancestry generalization, significantly increasing prediction accuracy in diverse populations by capturing both shared and ancestry-specific genetic signals through multi-ancestry training.

Further, these technical architectural innovations yield a significant and measurable improvement in disease prediction accuracy over existing methodologies. The embodiments herein demonstrate statistically significant performance gains (higher AUROC) over state-of-the-art summary-statistic-based methods in at least 11 out of 18 tested conditions and consistently outperform linear models across nearly all diseases. This enhanced efficacy is particularly pronounced at million-individual sample scales, where the non-linear transformer architecture captures subtle genetic interaction effects that are inherently undetectable by linear models.

Other technical improvements may also flow from these embodiments, and other technical problems may be solved. Thus, this statement of technical improvements is not limiting and instead constitutes examples of advantages that can be realized from the embodiments.

### VII. Appendix A: Details of the GWAS Runs

The internal ancestry classifier assigns individuals to one of five major genetic ancestry groups, African American (AFR), European (EUR), East Asian (EAS), South Asian (SAS), or Latino (LAT), based on local ancestry inference. To reduce confounding introduced by population structure, GWAS analyses were stratified by these genetically inferred ancestry groups. Principal component analysis (PCA) was conducted separately within each ancestry group using a subset of <100,000 high-quality genotyped variants shared across all internal platforms. A randomly selected subset of individuals was used for each group: 513K for AFR, 398K for EAS, 1M for EUR, 498 1M for LAT, and 111K for SAS. For each disease and ancestry group, GWAS was performed using logistic regression with additive allelic effects as predictors. Covariates included age, sex, genotype platform (to adjust for batch effects), and top principal components: specifically, the top 5 principal components for EUR, EAS, and SAS; the top 6 for AFR; and the top 9 for LAT. Association p-values were derived using a likelihood ratio test, comparing a reduced model fitted using covariates only to a full model fitted with both additive genetic effects and covariates.

### VIII. Appendix B: Details of LDpred2 Runs

For each disease, the Gibbs sampler LDpred2 software was used on the summary statistics with an internal LD panel. LD matrix computation included variants with minor allele frequency greater than 0.1%, and genotype call rate greater than 90%. Variants greater than 5 cM apart were assumed to be independent. Summary statistics were filtered to keep variants that had a minor allele frequency greater than 0.1% and had a genotype call rate greater than 95%. This consisted of variant sets with roughly 445,000 variants. Posterior SNP-effect sizes using the grid option was estimated with a set of 100 combinations of hyperparameters, leading to up to 100 sets of PRS per disease (depending on convergence). The hyperparameters that LDpred2 takes are an estimate for the proportion of causal variants, p, and trait heritability, h². LD score regression was used to estimate h², then used a grid of the h² estimate multiplied by 0.6, 0.8, 1, 1.2, and 1.4. For p, a sequence of values equally spaced on a logarithmic scale from 10⁻⁵ to 1 was used. The best hyperparameters for each disease were selected based on validation AUROC leading to the final LDPred2 PRS models. For Stacked LDPred2, however, all of the converged PRSs per disease (up to 100) were assembled by training elastic net on the validation data using 5-fold cross validation.

### IX Appendix C: Subsetting Variants for Downsampled Experiments

When training on smaller datasets, the same high-powered variant selection may not be available as in the full-data setting. To account for this, variant selection using GWAS was repeated and summary statistics adjusted to reflect the reduced sample size of each downsampled dataset. For each downsampled dataset, GWAS p-values were estimated under the reduced sample size using the original GWAS summary statistics.

$$Z = \frac{β}{SE} ; {Z}_{ds} = Z \times \sqrt{\frac{{N}_{ds}}{N}} ; {p}_{ds} = 2 Φ ( - \vert {Z}_{ds} \vert )$$

Where β and SE denote the effect size and standard error from the original GWAS, N is the original sample size, N_{ds} is the downsampled sample size, and Φ is the standard normal cumulative distribution function. Subsequently, variant selection was performed independently for each disease and each downsampled dataset using the estimated p-values, applying a threshold of p<0.01. Multitask model training was then conducted using the union of the selected variant sets across diseases at each downsampled scale, following the same procedure as in the full-data experiments. Additional details on variant sets, data sizes, and model configurations are provided in FIGS. 9A-9J.

### X. Supplementary Figures

FIGS. 8A-1, 8A-2, and 8A-3 illustrate benchmarking PRSformer against baseline methods using AUPRC as the evaluation metric. Numbers above the bars indicate test set AUPRC values; error bars denote 95% confidence intervals estimated via bootstrapped test samples. The reported p-value reflects the one-sided statistical significance of PRSformer outperforming LDPred2.

FIGS. 8B-1 and 8B-2 illustrate benchmarking of models trained on the combined training and validation datasets. Numbers above the bars indicate test set AUROC; error bars represent 95% confidence intervals computed via bootstrapped test samples. The two sets of p-values reflect the one-sided statistical significance of PRSformer+outperforming stacked LDPred2, and PRSformer outperforming non-stacked LDPred2, the latter being the same as those reported in FIGS. 4A and 4B.

FIGS. 8C-1 and 8C-2 illustrate prediction performance across different training scales for the non-linear model and linear baseline, both trained in a multitask setting using the same input variant set as PRSFormer. For most diseases, performance improves with more training data, with the non-linear model surpassing the linear baseline at larger scales. Fluctuations in performance for polymyalgia rheumatica and axial spondyloarthritis likely stem from the former's rarity and the latter's relatively small training size.

FIGS. 8D-1 and 8D-2 illustrate prediction performance across different training scales for the non-linear model and linear baseline, both trained in a multitask setting using subsetted variant sets at each downsampled scale. For most diseases, performance improves with increasing training data, with the non-linear model outperforming the linear baseline at larger scales. These trends are consistent with those observed using a fixed input variant set across scales.

FIG. 8E illustrates comparison of multitask versus single-task training for three additional diseases across different training scales, all using the same input variant set as PRSformer. X-axis values outside parentheses indicate single-task training sizes, and those inside indicate corresponding multitask training sizes. Across all tested diseases, multitask training outperforms single-task training for both the non-linear model and the linear baseline.

FIGS. 8F-1 and 8F-2 illustrate comparison of multitask versus single-task training for four tested diseases across different training scales. Both multitask and single-task models were trained on downsampled datasets using subsetted variants; additionally, single-task models used disease-specific variant sets. X-axis values outside parentheses indicate single-task training sizes, while those inside indicate the corresponding multitask sizes. Across all tested diseases, multitask training outperforms single-task training for both the non-linear model and the linear baseline. These results are consistent with those observed using a fixed input variant set for both multitask and single-task across scales.

FIG. 9A illustrates the number of selected variants per disease across European datasets.

FIG. 9B illustrates training sample sizes (case/control) per disease across European and multi-ancestry datasets.

FIG. 9C illustrates test data sample sizes (case/control) per disease across ancestry groups.

FIG. 9D illustrates validation dataset sample sizes (case/control) per disease (only European individuals).

FIG. 9E illustrates tuning of the training steps based on validation AUROC. Most models (e.g., model-t059) showed signs of overfitting beyond 2 training epochs. Training durations around this point (1.8-2.2 epochs) were explored and two epochs were selected as the final configuration.

FIG. 9F illustrates tuning of attention heads and model dimension based on validation AUROC. Four attention heads with d_{model}=64 were selected for their competitive performance despite a smaller model dimension compared to model-t21.

FIG. 9G illustrates tuning of attention dilation and number of transformer blocks based on validation AUROC. Increasing the number of transformer blocks and applying dilated attention (e.g., (1-2), (1-2-3)) led to mild overfitting. Two transformer blocks without dilation (1-1) were selected as the final configuration.

FIG. 9H illustrates tuning of the window size of neighborhood attention based on validation AUROC. Four attention heads were selected with a neighborhood attention window size of 385 as the final configuration.

FIG. 9I illustrates the number of selected variants per disease across non-European ancestries.

FIG. 9J illustrates characteristics of multitask models trained on down-sampled datasets with subsetted input variants.

### XI. Example Operations

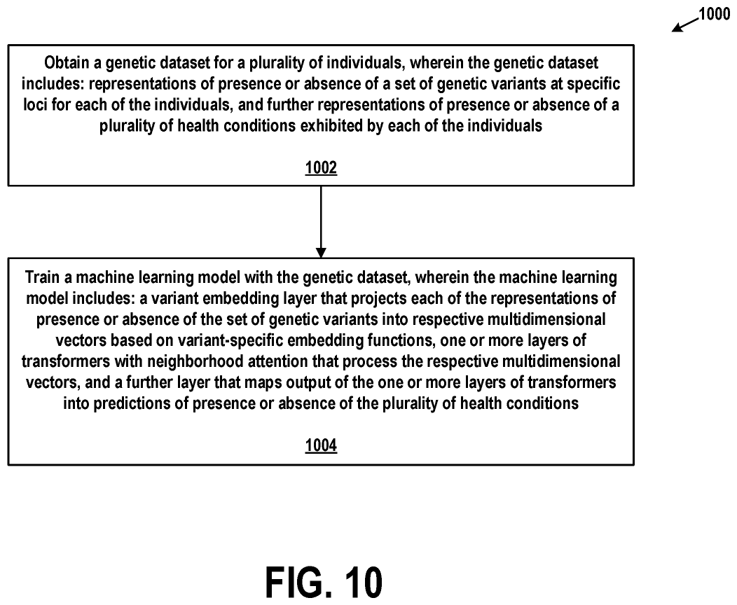

FIG. 10 is a flow chart 1000 illustrating an example embodiment. The process illustrated by FIG. 10 may be carried out by a computing device, such as computing device 100, and/or a cluster of computing devices, such as server cluster 200. However, the process can be carried out by other types of devices or device subsystems. For example, the process could be carried out by a computational instance of a remote network management platform or a portable computer, such as a laptop or a tablet device.

The embodiments of FIG. 10 may be simplified by the removal of any one or more of the features shown therein. Further, these embodiments may be combined with features, aspects, and/or implementations of any of the previous figures or otherwise described herein.

Block 1002 may involve obtaining a genetic dataset for a plurality of individuals, wherein the genetic dataset includes: representations of presence or absence of a set of genetic variants at specific loci for each of the individuals, and further representations of presence or absence of a plurality of health conditions exhibited by each of the individuals.

Block 1004 may involve training a machine learning model with the genetic dataset, wherein the machine learning model includes: a variant embedding layer that projects each of the representations of presence or absence of the set of genetic variants into respective multidimensional vectors based on variant-specific embedding functions, one or more layers of transformers with neighborhood attention that process the respective multidimensional vectors, and a further layer that maps output of the one or more layers of transformers into predictions of presence or absence of the plurality of health conditions.

In some embodiments, the plurality of individuals exceeds 100,000, wherein the set of genetic variants exceeds 10,000.

In some embodiments, the plurality of individuals exceeds 1,000,000, wherein the set of genetic variants exceeds 100,000.

In some embodiments, the representations of presence or absence of the set of genetic variants indicate whether each of the genetic variants is not present in either chromosome, heterozygous, homozygous, or missing a genotype.

In some embodiments, the variant embedding layer utilizes a first learned variant-specific embedding for whether each of the genetic variants is not present in either chromosome, heterozygous, or homozygous, wherein the variant embedding layer uses a second learned variant-specific embedding for whether each of the genetic variants is missing a genotype.

In some embodiments, the further representations of presence or absence of the plurality of health conditions are binary indicators.

In some embodiments, the respective multidimensional vectors are between 32 and 96 elements, inclusive.

In some embodiments, the neighborhood attention employs a sliding window of a size between 300 and 500 tokens of the machine learning model, inclusive.

In some embodiments, the neighborhood attention restricts token attention of the machine learning model to a local genomic window of a size between 300 and 500 tokens to achieve linear computational and memory complexity, thereby enabling processing of 100,000 or more genetic variants that are computationally infeasible for standard self-attention mechanisms.

In some embodiments, the one or more layers of transformers are stacked such that initial layers model genetic interactions within LD blocks and subsequent layers integrate information across neighboring LD blocks to capture longer-range genomic patterns influencing diseases.

In some embodiments, training the machine learning model involves use of a loss function that results in: small values when the predictions of presence or absence of the plurality of health conditions agree with the further representations of presence or absence of the plurality of health conditions, and larger values when the predictions of presence or absence of the plurality of health conditions do not agree with the further representations of presence or absence of the plurality of health conditions.

In some embodiments, the genetic dataset does not include entire genome wide association summary statistic study data.

In some embodiments, the genetic dataset includes populations of individuals from multiple distinct geographic regions.

In some embodiments, the predictions of the plurality of health conditions are generated simultaneously using shared variant embeddings and shared transformer blocks.

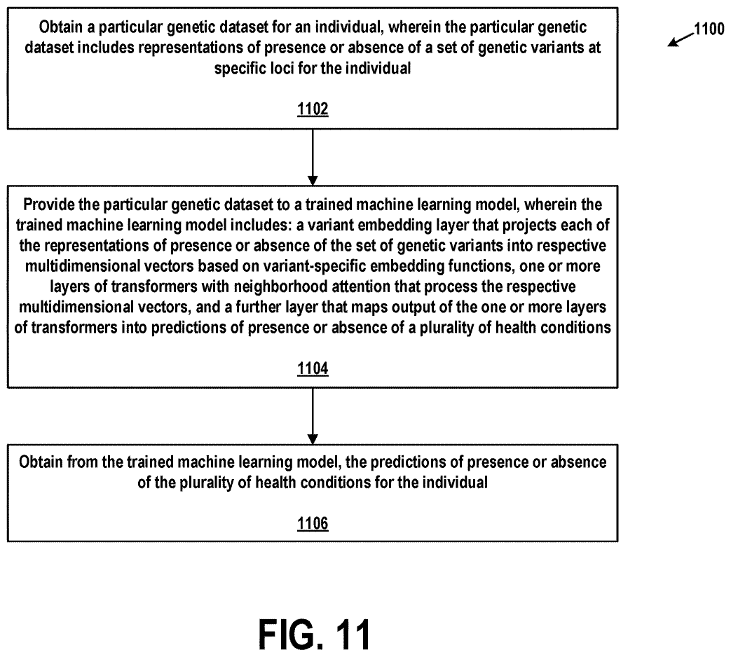

FIG. 11 is a flow chart 1100 illustrating an example embodiment. The process illustrated by FIG. 11 may be carried out by a computing device, such as computing device 100, and/or a cluster of computing devices, such as server cluster 200. However, the process can be carried out by other types of devices or device subsystems. For example, the process could be carried out by a computational instance of a remote network management platform or a portable computer, such as a laptop or a tablet device.

The embodiments of FIG. 11 may be simplified by the removal of any one or more of the features shown therein. Further, these embodiments may be combined with features, aspects, and/or implementations of any of the previous figures or otherwise described herein.

Block 1102 may involve obtaining a particular genetic dataset for an individual, wherein the particular genetic dataset includes representations of presence or absence of a set of genetic variants at specific loci for the individual.

Block 1104 may involve providing the particular genetic dataset to a trained machine learning model, wherein the trained machine learning model includes: a variant embedding layer that projects each of the representations of presence or absence of the set of genetic variants into respective multidimensional vectors based on variant-specific embedding functions, one or more layers of transformers with neighborhood attention that process the respective multidimensional vectors, and a further layer that maps output of the one or more layers of transformers into predictions of presence or absence of a plurality of health conditions.

Block 1106 may involve obtaining from the trained machine learning model, the predictions of presence or absence of the plurality of health conditions for the individual.

Some embodiments may involve providing, for display on a graphical user interface, the predictions of presence or absence of one or more of the health conditions for the individual.

In some embodiments, the representations of presence or absence of the set of genetic variants indicate whether each of the genetic variants is not present in either chromosome, heterozygous, homozygous, or of an unknown arrangement.

In some embodiments, the neighborhood attention restricts token attention of the trained machine learning model to a local genomic window of a size between 300 and 500 tokens to achieve linear computational and memory complexity, thereby enabling processing of 100,000 or more genetic variants that are computationally infeasible for standard self-attention mechanisms.

In some embodiments, the variant embedding layer utilizes a first learned variant-specific embedding for whether each of the genetic variants is not present in either chromosome, heterozygous, or homozygous, wherein the variant embedding layer uses a second learned variant-specific embedding for whether each of the genetic variants is missing a genotype.

The related embodiments of FIGS. 10 and 11 both provide a specific, technical improvement over previous solutions. Particularly, they both address the scalability bottleneck inherent in standard transformer architectures by replacing prohibitive quadratically scaling self-attention mechanisms with neighborhood attention. This architectural modification achieves linear complexity per layer, making it computationally feasible to process genome-scale inputs exceeding 100,000 variants without the memory and processor overhead that characterizes traditional genomic sequence modeling. Unlike prior methods that rely on summary statistics and discard individual-level haplotype data, these embodiments utilize a variant embedding layer and stack neighborhood attention layers to capture complex, non-linear genetic interactions, such as epistasis, directly from raw genotypes. This specific deep learning framework restricts attention initially to local genomic windows, which allows the model to capture dependencies within LD blocks while maintaining the statistical power necessary to detect subtle patterns at a million-individual scale.

### XII. Closing

The present disclosure is not to be limited in terms of the particular embodiments described in this application, which are intended as illustrations of various aspects. Many modifications and variations can be made without departing from its scope, as will be apparent to those skilled in the art. Functionally equivalent methods and apparatuses within the scope of the disclosure, in addition to those described herein, will be apparent to those skilled in the art from the foregoing descriptions. Such modifications and variations are intended to fall within the scope of the appended claims.

The above detailed description describes various features and operations of the disclosed systems, devices, and methods with reference to the accompanying figures. The example embodiments described herein and in the figures are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations.

With respect to any or all of the message flow diagrams, scenarios, and flow charts in the figures and as discussed herein, each step, block, and/or communication can represent a processing of information and/or a transmission of information in accordance with example embodiments. Alternative embodiments are included within the scope of these example embodiments. In these alternative embodiments, for example, operations described as steps, blocks, transmissions, communications, requests, responses, and/or messages can be executed out of order from that shown or discussed, including substantially concurrently or in reverse order, depending on the functionality involved. Further, more or fewer blocks and/or operations can be used with any of the message flow diagrams, scenarios, and flow charts discussed herein, and these message flow diagrams, scenarios, and flow charts can be combined with one another, in part or in whole.

A step or block that represents a processing of information can correspond to circuitry that can be configured to perform the specific logical functions of a herein-described method or technique. Alternatively or additionally, a step or block that represents a processing of information can correspond to a module, a segment, or a portion of program code (including related data). The program code can include one or more instructions executable by a processor for implementing specific logical operations or actions in the method or technique. The program code and/or related data can be stored on any type of non-transitory computer readable medium such as a storage device including RAM, ROM, a disk drive, a solid-state drive, or another tangible storage medium.

Moreover, a step or block that represents one or more information transmissions can correspond to information transmissions between software and/or hardware modules in the same physical device. However, other information transmissions can be between software modules and/or hardware modules in different physical devices.

The particular arrangements shown in the figures should not be viewed as limiting. It should be understood that other embodiments could include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purpose of illustration and are not intended to be limiting, with the true scope being indicated by the following claims.

What is claimed is:

1. A computer-implemented method comprising:
obtaining a genetic dataset for a plurality of individuals, wherein the genetic dataset includes: representations of presence or absence of a set of genetic variants at specific loci for each of the individuals, and further representations of presence or absence of a plurality of health conditions exhibited by each of the individuals; and
training a machine learning model with the genetic dataset, wherein the machine learning model includes: a variant embedding layer that projects each of the representations of presence or absence of the set of genetic variants into respective multidimensional vectors based on variant-specific embedding functions, one or more layers of transformers with neighborhood attention that process the respective multidimensional vectors, and a further layer that maps output of the one or more layers of transformers into predictions of presence or absence of the plurality of health conditions.

2. The computer-implemented method of claim 1, wherein the plurality of individuals exceeds 100,000, and wherein the set of genetic variants exceeds 10,000.

3. The computer-implemented method of claim 1, wherein the plurality of individuals exceeds 1,000,000, and wherein the set of genetic variants exceeds 100,000.

4. The computer-implemented method of claim 1, wherein the representations of presence or absence of the set of genetic variants indicate whether each of the genetic variants is not present in either chromosome, heterozygous, homozygous, or missing a genotype.

5. The computer-implemented method of claim 1, wherein the variant embedding layer utilizes a first learned variant-specific embedding for whether each of the genetic variants is not present in either chromosome, heterozygous, or homozygous, and wherein the variant embedding layer uses a second learned variant-specific embedding for whether each of the genetic variants is missing a genotype.

6. The computer-implemented method of claim 1, wherein the further representations of presence or absence of the plurality of health conditions are binary indicators.

7. The computer-implemented method of claim 1, wherein the respective multidimensional vectors are between 32 and 96 elements, inclusive.

8. The computer-implemented method of claim 1, wherein the neighborhood attention employs a sliding window of a size between 300 and 500 tokens of the machine learning model, inclusive.

9. The computer-implemented method of claim 1, wherein the neighborhood attention restricts token attention of the machine learning model to a local genomic window of a size between 300 and 500 tokens to achieve linear computational and memory complexity, thereby enabling processing of 100,000 or more genetic variants that are computationally infeasible for standard self-attention mechanisms.

10. The computer-implemented method of claim 1, wherein the one or more layers of transformers are stacked such that initial layers model genetic interactions within linkage disequilibrium blocks and subsequent layers integrate information across neighboring linkage disequilibrium blocks to capture longer-range genomic patterns influencing diseases.

11. The computer-implemented method of claim 1, wherein training the machine learning model involves use of a loss function that results in: small values when the predictions of presence or absence of the plurality of health conditions agree with the further representations of presence or absence of the plurality of health conditions, and larger values when the predictions of presence or absence of the plurality of health conditions do not agree with the further representations of presence or absence of the plurality of health conditions.

12. The computer-implemented method of claim 1, wherein the genetic dataset does not entire include genome-wide association summary statistic study data.

13. The computer-implemented method of claim 1, wherein the genetic dataset includes populations of individuals from multiple distinct geographic regions.

14. The computer-implemented method of claim 1, wherein the predictions of the plurality of health conditions are generated simultaneously using shared variant embeddings and shared transformer blocks.

15. A computer-implemented method comprising:
obtaining a particular genetic dataset for an individual, wherein the particular genetic dataset includes representations of presence or absence of a set of genetic variants at specific loci for the individual;
providing the particular genetic dataset to a trained machine learning model, wherein the trained machine learning model includes: a variant embedding layer that projects each of the representations of presence or absence of the set of genetic variants into respective multidimensional vectors based on variant-specific embedding functions, one or more layers of transformers with neighborhood attention that process the respective multidimensional vectors, and a further layer that maps output of the one or more layers of transformers into predictions of presence or absence of a plurality of health conditions; and
obtaining from the trained machine learning model, the predictions of presence or absence of the plurality of health conditions for the individual.

16. The computer-implemented method of claim 15, further comprising:
providing, for display on a graphical user interface, the predictions of presence or absence of one or more of the health conditions for the individual.

17. The computer-implemented method of claim 15, wherein the representations of presence or absence of the set of genetic variants indicate whether each of the genetic variants is not present in either chromosome, heterozygous, homozygous, or of an unknown arrangement.

18. The computer-implemented method of claim 15, wherein the neighborhood attention restricts token attention of the trained machine learning model to a local genomic window of a size between 300 and 500 tokens to achieve linear computational and memory complexity, thereby enabling processing of 100,000 or more genetic variants that are computationally infeasible for standard self-attention mechanisms.

19. The computer-implemented method of claim 15, wherein the variant embedding layer utilizes a first learned variant-specific embedding for whether each of the genetic variants is not present in either chromosome, heterozygous, or homozygous, and wherein the variant embedding layer uses a second learned variant-specific embedding for whether each of the genetic variants is missing a genotype.

20. A non-transitory computer-readable medium, having stored thereon program instructions that, upon execution by a computing system, cause the computing system to perform operations comprising:
obtaining a genetic dataset for a plurality of individuals, wherein the genetic dataset includes: representations of presence or absence of a set of genetic variants at specific loci for each of the individuals, and further representations of presence or absence of a plurality of health conditions exhibited by each of the individuals; and
training a machine learning model with the genetic dataset, wherein the machine learning model includes: a variant embedding layer that projects each of the representations of presence or absence of the set of genetic variants into respective multidimensional vectors based on variant-specific embedding functions, one or more layers of transformers with neighborhood attention that process the respective multidimensional vectors, and a further layer that maps output of the one or more layers of transformers into predictions of presence or absence of the plurality of health conditions.
